(19) United States
(12) Patent Application Publication
(10) Pub. No.: US 2009/0036388 A1
(43) Pub. Date: Feb. 5, 2009

(54) Compounds

(76) Inventors: Sulejman Alihodzic, Zagreb (HR); Mihaela Peric, Zagreb (HR); Dijana Pesic, Zagreb (HR)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(21) Appl. No.: 12/180,675

(22) Filed: Jul. 28, 2008

Related U.S. Application Data
(60) Provisional application No. 60/952,649, filed on Jul. 30, 2007.

(51) Int. Cl. C07H 17/08; A61K 31/7048; A61P 33/06

(52) U.S. Cl. 514/29; 536/7.4

(57) ABSTRACT

The present invention relates to novel 2′-O-substituted 9-deoxo-9a-methyl-9a-aza-9a-homoerythromycin A derivatives having antimalarial activity. More particularly, the invention relates to 2′-O-substituted-9-deoxo-9a-methyl-9a-aza-9a-homoerythromycin A and 2′-O-substituted-3-O-decladinosyl-9-deoxo-9a-methyl-9a-aza-9a-homoerythromycin A derivatives having antimalarial activity, to the intermediates for their preparation, to the methods for their preparation, to their use as therapeutic agents, and to salts thereof having antimalarial activity.

### FIELD OF THE INVENTION

The present invention relates to novel 2′-O-substituted 9-deoxo-9a-methyl-9a-aza-9a-homoerythromycin A derivatives having antimalarial activity. More particularly, the invention relates to 2′-O-substituted-9-deoxo-9a-methyl-9a-aza-9a-homoerythromycin A and 2′-O-substituted-3-O-decladinosyl-9-deoxo-9a-methyl-9a-aza-9a-homoerythromycin A having antimalarial activity, to the intermediates for their preparation, to the methods for their preparation, to their use as therapeutic agents, and to salts thereof having antimalarial activity.

### BACKGROUND OF THE INVENTION

Malaria is a serious infection. 200 to 300 million people are infected with malaria and two to three million people die from malaria every year. The disease is caused by a parasite (a protozoa of the Plasmodia genus), which is transmitted by the female Anopheles mosquito. There are four parasites that can affect humans, Plasmodium falciparum, P. vivax, P. ovale, and P. malariae. A distinction is drawn between Malaria tropica (caused by Plasmodium falciparum), Malaria tertiana (caused by Plasmodium vivax or Plasmodium ovale) and Malaria quartana (caused by Plasmodium malariae). Malaria tropica is the most severe form of the disease, and is characterized by severe constitutional symptoms, and sometimes causes death.

Malaria is characterized by attacks of chills, fever, and sweating, occurring at intervals which depend on the time required for development of a new generation of parasites in the body. After recovery from the acute attack, the disease has a tendency to become chronic, with occasional relapses. The disease is prevalent in tropical and subtropical areas of the world including the Amazon region of Brazil, East and Southern Africa and Southeast Asia. The emergence of a malaria parasite resistant to chloroquine, which is a drug used extensively in the treatment of malaria, has become a serious problem, and therefore, there is an urgent need to develop an effective remedy. Also, attempts to develop a malaria vaccine have failed to date. This compounds the urgent need to find an alternative drug-based approach to treating malaria.

Drugs of diverse chemical classes, such as chloroquine, mefloquine, halofantrine, and artemisinin, atovaquone/proguanil (Malarone™), doxycycline, and primaquine have been developed for the treatment of malaria. However, while marginally successful against some strains of malaria, most strains of malaria appear to have developed resistance not only to individual drugs but also to multiple combinations of drugs. Drugs which worked initially become totally ineffective after a period of time. An initial period of remission is often followed by a period during which nothing seems to be effective against the disease. This is known as multiple drug resistance, and it remains an issue in antimalarial drug development efforts. A malarial parasite which initially responds to treatment by one or more drugs becomes resistant to treatment not only using the drugs previously used, but many other antimalarial drugs. This further underscores the urgent necessity to find new compounds which show good efficacy against malaria and minimal toxicity.

In recent years several reports indicated that macrolides have potential for prophylactic as well as therapeutic use against malaria. Midecamycinin was studied in 1989 in two infectious models using Plasmodium berghei and Plasmodium yoelii nigeriensis (mouse) and Plasmodium cynomolgi (rhesus monkey) [S. K. Puri and G. P. Duti, Chemotherap. 35 (1989) 187]. In both mouse models, the macrolide midecamycinin was active. The doses for Plasmodium berghei infection were significantly lower than for Plasmodium yoelii nigeriensis. In the monkey model, no efficacy was noted. In other investigations the animal model was challenged with azithromycin [S. K. Puri and N. Singh, Exp. Parasitol. 94 (2000) 8]. The dose regimen of 25-50 mg/kg reflects the same dose used for antibacterial treatment. Azithromycin worked in prophylactic and therapeutic dosing and in contrast to midecamycinin azithromycin was active also in the monkey model.

The efficacy of azithromycin in treating malarial infections was studied in Gambia [S. T. Sadiq et al, Lancet 346 (1995), 881]. Children undergoing therapy for trachoma (Azithromycin is highly effective against C. trachomatis) were also examined for signs of malaria prophylaxis or therapeutic effects. A clear improvement of various indicators of malaria infection suggested a significant therapeutic benefit of azithromycin. The prophylactic efficacy of azithromycin was confirmed in Kenya [S. L. Anderson et al., Ann. Intern. Med. 123 (1995) 771]. A significant protection in adult volunteers was achieved with a better prophylaxis obtained through use of a daily dosing scheme of 250 mg versus a weekly regimen of 1000 mg. Also, in a double-blind, placebo-controlled trial with azithromycin in Irian Jaya in Indonesia [W. R. Taylor et al., Clin. Infect. Dis. 28 (1999) 74], the prophylactic efficacy in azithromycin treated non-immune patients was 71.6% for Plasmodium falciparum and 98.9% for Plasmodium vivax as compared to controls.

### SUMMARY OF THE INVENTION

The present invention relates to novel 2′-O-substituted-9-deoxo-9a-methyl-9a-aza-9a-homoerythromycin A and 2′-substituted-3-O-decladinosyl-9-deoxo-9a-methyl-9a-aza-9a-homoerythromycin A derivatives represented by Formula (I):

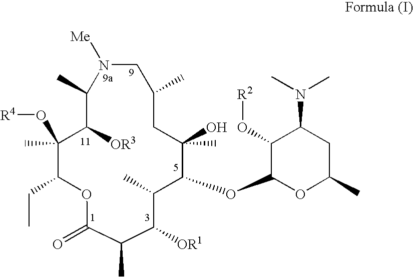

wherein
R¹ represents H or a α-L-cladinosyl group of Formula (a)

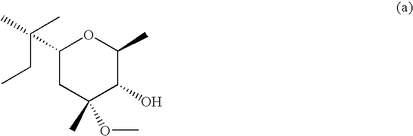

R² represents the formula —(CH₂)ₐ—X—(CH₂)_{b}—(NH)_{c}-A;
R³ represents H or —C(O)C₁₋₃alkyl or R³ and R⁴ taken together with the intervening atoms form a cyclic carbonate group of Formula (b):

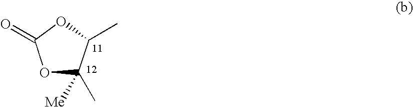

R⁴ represents H or R³ and R⁴ taken together with the intervening atoms form a cyclic carbonate group of Formula (b);
X represents —N(R⁵)—, —NHC(O)— or —C(O)NH—;
R⁵ represents H or C₁₋₃alkyl;
A represents a moiety of Formula (c) or (d):

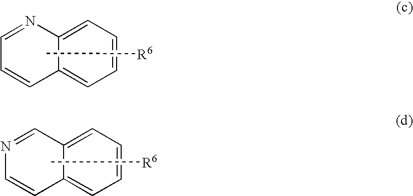

attached to the rest of the molecule through any available carbon atom;
R⁶ represents H or halogen and is attached to Formula (c) or (d) at any available carbon atom;
a is an integer from 2 to 6;
b is an integer from 0 to 6;
c is 0 or 1;
provided that when c is 1 then b is an integer from 1 to 6;
or salts thereof.

The present invention also relates to intermediates of Formula (II), useful for the preparation of compounds of Formula (I)

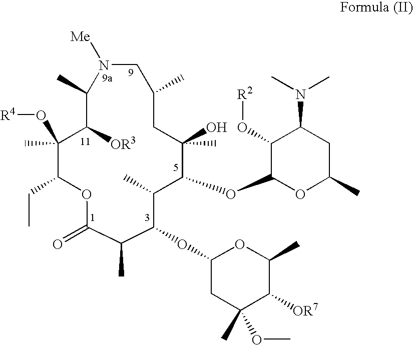

wherein
R² is aminopropyl;

### R³ is H or C(O)CH₃;

R⁴ represents H;
R⁷ is H or 3-aminopropyl.

The present invention also relates to pharmaceutical compositions comprising a compound of Formula (I) or a pharmaceutically acceptable salt thereof.

Furthermore, the present invention also relates to methods of treating malarial diseases comprising administration of a therapeutically effective amount of a compound of Formula (I) to a patient in need thereof. Moreover, novel compounds of Formula (I) of the present invention may exhibit good potency against plasmodia, especially against multiresistant plasmodial species.

According to another aspect of the invention there is provided a compound of Formula (I) or a pharmaceutically acceptable salt thereof for use in human or veterinary medical therapy.

According to another aspect of the invention there is provided a compound of Formula (I) or a pharmaceutically acceptable salt thereof for use in the therapeutic and/or prophylactic treatment of malaria.

In another aspect of the invention there is provided the use of a compound of Formula (I) or a pharmaceutically acceptable salt thereof in the manufacture of a medicament for the treatment of malaria.

In one aspect of the invention, the treatment is therapeutic or prophylactic treatment.

The present invention is also directed to compositions comprising a compound of Formula (I) or a pharmaceutically acceptable salt thereof in an amount effective for therapeutic and/or prophylactic treatment of malaria in a subject in need of such treatment.

The present invention is also directed to a method for using the compounds of Formula (I) in the prophylaxis of malaria or the treatment of subjects exposed to the malaria parasites.

### DETAILED DESCRIPTION OF THE INVENTION

In one particular embodiment, the present invention is directed to the novel 2′-substituted-9-deoxo-9a-methyl-9a-aza-9a-homoerythromycin A and 2′-substituted-3-O-decladinosyl-9-deoxo-9a-methyl-9a-aza-9a-homoerythromycin A derivatives represented by Formula (I):

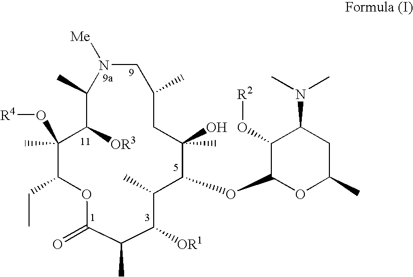

wherein
R¹ represents H or a α-L-cladinosyl group of Formula (a)

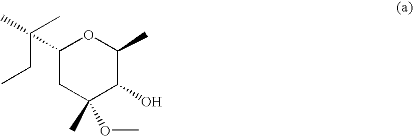

R² represents the formula —(CH₂)ₐ—X—(CH₂)_{b}—(NH)_{c}-A;
R³ represents H or —C(O)C₁₋₃alkyl or R³ and R⁴ taken together with the intervening atoms form a cyclic carbonate group of Formula (b)

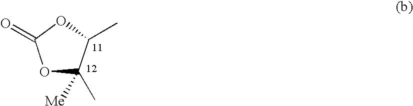

R⁴ represents H or R³ and R⁴ taken together with the intervening atoms form a cyclic carbonate group of Formula (b);
X represents —N(R⁵)—, —NHC(O)— or —C(O)NH—;
R⁵ represents H or C₁₋₃alkyl;
A represents a moiety of Formula (c) or (d):

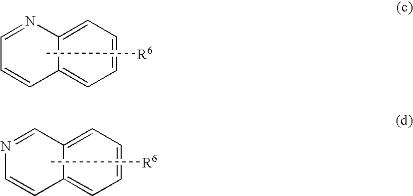

attached to the rest of the molecule through any available carbon atom;
R⁶ represents H or halogen and is attached to Formula (c) or (d) at any available carbon atom;
a is an integer from 2 to 6;
b is an integer from 0 to 6;
c is 0 or 1;
provided that when c is 1 then b is an integer from 1 to 6;
or salts thereof.

In a further embodiment the present invention also relates to intermediates of Formula (II), useful for the preparation of compounds of Formula (I)

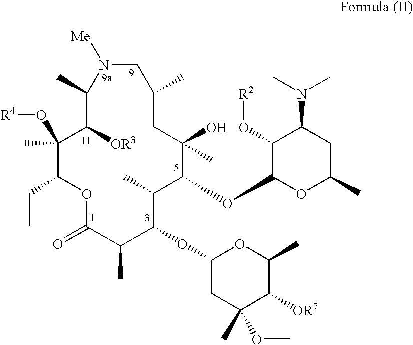

wherein
R² is aminopropyl

### R³ is H or C(O)CH₃;

R⁴ represents H;
R⁷ is H or 3-aminopropyl.

The phrase “pharmaceutically acceptable”, as used in connection with compositions of the invention, refers to molecular entities and other ingredients of such compositions that are physiologically tolerable and do not typically produce untoward reactions when administered to a mammal (e.g., human). Preferably, as used herein, the term “pharmaceutically acceptable” means approved by a regulatory agency of the Federal or a state government or listed in the U.S. Pharmacopoeia or other generally recognized pharmacopoeia for use in mammals, and more particularly in humans.

The term “carrier” applied to pharmaceutical compositions of the invention refers to a diluent, excipient, or vehicle with which an active compound is administered. Such pharmaceutical carriers can be sterile liquids, such as water, saline solutions, aqueous dextrose solutions, aqueous glycerol solutions, and oils, including those of petroleum, animal, vegetable or synthetic origin, such as peanut oil, soybean oil, mineral oil, sesame oil and the like. Suitable pharmaceutical carriers are described in “Remington's Pharmaceutical Sciences” by E. W. Martin, 18th Edition, incorporated by reference. Particularly preferred for the present invention are carriers suitable for immediate-release, i.e., release of most or all of the active ingredient over a short period of time, such as 60 minutes or less, and make rapid absorption of the drug possible.

The compounds of the present invention may be in the form of and/or may be administered as a pharmaceutically acceptable salt. For a review on suitable salts see Berge et al., J. Pharm. Sci., 66 (1977) 1-19.

Typically, a pharmaceutical acceptable salt may be readily prepared by using a desired acid. The salt may precipitate from solution and be collected by filtration or may be recovered by evaporation of the solvent. For example, an aqueous solution of an acid such as hydrochloric acid may be added to an aqueous suspension of a compound of formula (I) and the resulting mixture evaporated to dryness (lyophilised) to obtain the acid addition salt as a solid. Alternatively, a compound of formula (I) may be dissolved in a suitable solvent, for example an alcohol such as isopropanol, and the acid may be added in the same solvent or another suitable solvent. The resulting acid addition salt may then be precipitated directly, or by addition of a less polar solvent such as diisopropyl ether or hexane, and isolated by filtration.

Suitable addition salts are formed from inorganic or organic acids which form non-toxic salts and examples are hydrochloride, hydrobromide, hydroiodide, sulphate, bisulphate, nitrate, phosphate, hydrogen phosphate, acetate, trifluoroacetate, maleate, malate, fumarate, lactate, tartrate, citrate, formate, gluconate, succinate, pyruvate, oxalate, oxaloacetate, trifluoroacetate, saccharate, benzoate, alkyl or aryl sulphonates (eg methanesulphonate, ethanesulphonate, benzenesulphonate or p-toluenesulphonate) and isethionate. In one aspect of the invention, the salt is an acetate salt. Representative examples of salts include trifluoroacetate and formate salts, for example the bis or tris trifluoroacetate salts and the mono or diformate salts, in particular the tris or bis trifluoroacetate salt and the monoformate salt.

In another aspect compounds of the invention are pharmaceutically acceptable salts, solvates and esters. In a further aspect compounds of the invention are pharmaceutically acceptable salts and esters. In a further aspect compounds of the invention are pharmaceutically acceptable salts.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Solvates of the compounds of the invention are within the scope of the invention. The salts of the compound of Formula (I) may form solvates (e.g. hydrates) and the invention also includes all such solvates.

The present invention also relates to pharmaceutically acceptable esters of the compounds of Formula (I), for example carboxylic acid esters —COOR, in which R is selected from straight or branched chain alkyl, for example n-propyl, n-butyl, alkoxyalkyl (e.g. methoxymethyl), aralkyl (e.g. benzyl), aryloxyalkyl (e.g. phenoxymethyl), aryl (e.g. phenyl optionally substituted by halogen, C₁₋₄alkyl or C₁₋₄alkoxy or amino). Unless otherwise specified, any alkyl moiety present in such esters suitably contains 1 to 18 carbon atoms, particularly 1 to 4 carbon atoms. Any aryl moiety present in such esters suitably comprises a phenyl group.

References to a compound according to the invention include both compounds of Formula (I), and their pharmaceutically acceptable salts, solvates and esters.

With regard to stereoisomers, the compounds of Formula (I) have more than one asymmetric carbon atom. In the general Formula (I) as drawn, the solid wedge shaped bond indicates that the bond is above the plane of the paper. The broken bond indicates that the bond is below the plane of the paper.

It will be appreciated that the substituents on the macrolide may also have one or more asymmetric carbon atoms. Thus, the compounds of Formula (I) may occur as individual enantiomers or diastereomers. All such isomeric forms are included within the present invention, including mixtures thereof.

The present invention includes the individual stereoisomers of the compounds of the invention and, where appropriate, the individual stereoisomeric forms thereof, together with mixtures.

Separation of diastereoisomers may be achieved by conventional techniques, e.g. by fractional crystalisation, chromatography or HPLC An individual stereoisomer may also be prepared from a corresponding optically pure intermediate or by resolution, such as HPLC, of the corresponding mixture using a suitable chiral support or by fractional crystalisation of the diastereoisomeric salts formed by reaction of the corresponding mixture with a suitable optically active acid or base, as appropriate.

The compounds of Formula (I) may be in crystalline or amorphous form. Furthermore, some of the crystalline forms of the compounds of Formula (I) may exist as polymorphs, which are included in the present invention.

In one aspect of the invention R¹ represents H.

In one aspect of the invention R¹ represents an α-L-cladinosyl group of Formula (a).

In one aspect of the invention R² represents Formula —(CH₂)ₐ—X—(CH₂)_{b}—(NH)_{c}-A wherein X is —NHC(O)—, c is 1, and a, b and A are as defined in Formula (I) above.

In another aspect of the invention R² represents Formula —(CH₂)ₐ—X—(CH₂)_{b}—(NH)_{c}-A wherein X is —NHC(O)—, a is 3, b is 3, c is 1, and A is a moiety of Formula (c).

In one aspect of the invention R² represents Formula —(CH₂)ₐ—X—(CH₂)_{b}—(NH)_{c}-A wherein X is —NH—, a is 3, b is 0, c is 0, and A is a moiety of Formula (c).

In one aspect of the invention the sum of a and b is less than or equal to 8. In a further aspect of the invention the sum of a and b is 3, 4 or 6.

In one aspect of the invention a is 3, b is 1 and c is 1. In a further aspect of the invention a is 3, b is 1 and c is 0. In a further aspect of the invention a is 3, b is 0 and c is 0. In a further aspect of the invention a is 3, b is 3 and c is 1. In a further aspect of the invention a is 2, b is 2 and c is 1.

In one aspect of the invention, A is a moiety of Formula (c) attached to the rest of the molecule via the 2-, 3-, or 4-position. In a further aspect of the invention, A is a moiety of Formula (d) attached to the rest of the molecule via the 1-position.

In a further aspect of the invention A represents 2-quinolinyl, 3-quinolinyl, 4-quinolinyl, 7-chloro-4-quinolinyl or 3-chloro-1-isoquinolinyl. In a further aspect of the invention A represents 2-quinolinyl, 3-quinolinyl, 4-quinolinyl, or 7-chloro-4-quinolinyl. In a further aspect of the invention A represents 7-chloro-4-quinolinyl.

In one aspect of the invention R³ represents H.

In one aspect of the invention R⁴ represents H.

In one aspect of the invention R⁵ represents H or methyl. In a further aspect of the invention R⁵ represents H.

In one aspect of the invention R⁶ represents H. In a further aspect of the invention R⁶ represents a chlorine atom. In a further aspect of the invention R⁶ represents a chlorine atom attached to the moiety of Formula (c) in the 7-position. In a further aspect of the invention R⁶ represents a chlorine atom attached to the moiety of Formula (d) in the 3-position.

It will be understood that the present invention covers all combinations of aspects, suitable, convenient and preferred groups described herein.

The term “alkyl” as used herein, refers to a saturated, straight or branched-chain hydrocarbon radical containing the stated number of carbon atoms, for example C₁₋₃alkyl contains between one and three carbon atoms. Examples of “C₁₋₃alkyl” radicals include; methyl, ethyl, propyl, isopropyl.

The term “lower alcohol”, as used herein, refers to a C₁₋₄alcohol, such as for example, methanol, ethanol, propanol, isopropanol, butanol, t-butanol, and the like.

The term “halogen” refers to a fluorine, chlorine, bromine or iodine atom.

The term “inert solvent”, as used herein, refers to a solvent that cannot react with the dissolved compounds including non-polar solvent such as hexane, toluene, diethyl ether, diisopropylether, chloroform, ethyl acetate, THF, dichloromethane; polar aprotic solvents such as acetonitrile, acetone, N,N-dimethylformamide, N,N-dimethylacetamide, dimethyl sulfoxide, pyridine, and polar protic solvents such as lower alcohol, acetic acid, formic acid and water.

Compounds of the Formula (I) include:
2′-O-[3-({4-[(7-chloro-4-quinolinyl)amino]butanoyl}amino)propyl]-9-deoxo-9a-methyl-9a-aza-9a-homoerythromycin A;
2′-O-[3-({4-[(7-chloro-4-quinolinyl)amino]butanoyl}amino)propyl]-3-O-decladinosyl-9-deoxo-9a-methyl-9a-aza-9a-homoerythromycin A;
2′-O-{3-[(7-Chloro-4-quinolinyl)amino]propyl}-9-deoxo-9a-methyl-9a-aza-9a-homoerythromycin A;
11-O-Acetyl-2′-O-{3-[(7-Chloro-4-quinolinyl)amino]propyl}-3-O-decladinosyl-9-deoxo-9a-methyl-9a-aza-9a-homoerythromycin A;
2′-O-{3-[(7-Chloro-4-quinolinyl)amino]propyl}-3-O-decladinosyl-9-deoxo-9a-methyl-9a-aza-9a-homoerythromycin A;
2′-O-{3-[(4-quinolinyl)amino]propyl}-9-deoxo-9a-methyl-9a-aza-9a-homoerythromycin A;
2′-O-[3-({2-[(7-chloro-4-quinolinyl)amino]ethanoyl}amino)propyl]-9-deoxo-9a-methyl-9a-aza-9a-homoerythromycin A;
2′-O-[3-({2-[(7-chloro-4-quinolinyl)amino]ethanoyl}amino)propyl]-3-O-decladinosyl-9-deoxo-9a-methyl-9a-aza-9a-homoerythromycin A;
2′-O-[3-({2-[(4-quinolinyl)amino]ethanoyl}amino)propyl]-9-deoxo-9a-methyl-9a-aza-9a-homoerythromycin A;
2′-O-[3-({2-[(4-quinolinyl)amino]ethanoyl}amino)propyl]-3-O-decladinosyl-9-deoxo-9a-methyl-9a-aza-9a-homoerythromycin A;
2′-O-[3-({2-[(7-chloro-4-quinolinyl)amino]ethyl}amino)-3-oxopropyl]9-deoxo-9a-methyl-9a-aza-9a-homoerythromycin A;
2′-O-[3-({4-[(4-quinolinyl)amino]butanoyl}amino)propyl]-9-deoxo-9a-methyl-9a-aza-9a-homoerythromycin A;
2′-O-{3-[(3-quinolinylcarbonyl)amino]propyl}-9-deoxo-9a-methyl-9a-aza-9a-homoerythromycin A;
2′-O-{3-[(4-quinolinylmethyl)amino]propyl}-9-deoxo-9a-methyl-9a-aza-9a-homoerythromycin A;
2′-O-{3-[(4-quinolinylmethyl)amino]propyl}-3-O-decladinosyl-9-deoxo-9a-methyl-9a-aza-9a-homoerythromycin A;
2′-O-{3-[methyl(4-quinolinylmethyl)amino]propyl}-9-deoxo-9a-methyl-9a-aza-9a-homoerythromycin A;
2′-O-{3-[(3-quinolinylmethyl)amino]propyl}-9-deoxo-9a-methyl-9a-aza-9a-homoerythromycin A;
2′-O-{3-[methyl(3-quinolinylmethyl)amino]propyl}-9-deoxo-9a-methyl-9a-aza-9a-homoerythromycin A;
2′-O-{3-[(3-quinolinylmethyl)amino]propyl}-3-O-decladinosyl-9-deoxo-9a-methyl-9a-aza-9a-homoerythromycin A;
2′-O-{3-[(2-quinolinylmethyl)amino]propyl}-9-deoxo-9a-methyl-9a-aza-9a-homoerythromycin A;
2′-O-{3-[(3-chloro-1-isoquinolinyl)amino]propyl}-9-deoxo-9a-methyl-9a-aza-9a-homoerythromycin A; and
2′-O-{3-[methyl(3-quinolinylmethyl)amino]propyl}-3-O-decladinosyl-9-deoxo-9a-methyl-9a-aza-9a-homoerythromycin A;
   and salts thereof.

Compounds of the Formula (II) include:
2′—O-(3-Aminopropyl)-9-deoxo-9a-methyl-9a-aza-9a-homoerythromycin A; and
11-O-Acetyl-2′-O, 4″-O-di-(3-aminopropyl)-9-deoxo-9a-methyl-9a-aza-9a-homo-erythromycin A;
   and salts thereof.

“Treating” or “Treatment” of malaria includes therapeutic treatment and prophylactic treatment.

“Therapeutic treatment” of malaria includes
i. preventing or delaying the appearance of clinical symptoms of malaria developing in a mammal that has been in contact with the parasite.
ii. inhibiting the malaria, i.e., arresting, reducing or delaying the development of malaria or a relapse thereof or at least one clinical or subclinical symptom thereof, or
iii. relieving or attenuating one or more of the clinical or subclinical symptoms of malaria.

The benefit to a subject to be treated is either statistically significant or at least perceptible to the patient or to the physician.

“Prophylactic treatment” or “prophylaxis” of malaria includes treating subjects who are at risk of developing malaria. This includes the treatment of subjects who have been exposed to malaria-bearing mosquitoes, the treatment of subjects who intend to travel to a country where malaria is endemic and the treatment of subjects who otherwise risk exposure to malaria-bearing mosquitoes.

“Maintenance therapy” is preventive therapy that follows successful initial treatment of the acute phase of the illness where regular (usually smaller) doses of the drug are delivered to the patient to prevent recurrence and worsening of the disease. The Plasmodium vivax and P. ovale parasites have dormant liver stages that can remain silent for years. Maintenance therapy for these strains is particularly important. The hallmarks of the acute phase include symptoms like chills and fever.

“Subject” refers to an animal, in particular a mammal and more particularly to a human or a domestic animal or an animal serving as a model for a disease (e.g., mouse, monkey, etc.). In one aspect, the subject is a human. As used herein, the term patient is used synonymously with subject.

A “therapeutically effective amount” means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated and will be ultimately at the discretion of the attendant physician.

### Pharmaceutical Compositions

While it is possible that, for use in the methods of the invention, a compound of Formula (I) may be administered as the bulk substance, it is preferable to present the active ingredient in a pharmaceutical formulation, for example, wherein the agent is in admixture with at least one pharmaceutically acceptable carrier selected with regard to the intended route of administration and standard pharmaceutical practice.

The term “carrier” refers to a diluent, excipient, and/or vehicle with which an active compound is administered. The pharmaceutical compositions of the invention may contain combinations of more than one carrier. Such pharmaceutical carriers can be sterile liquids, such as water, saline solutions, aqueous dextrose solutions, aqueous glycerol solutions, and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin, 18th Edition. The choice of pharmaceutical carrier can be selected with regard to the intended route of administration and standard pharmaceutical practice. The pharmaceutical compositions may comprise as, in addition to, the carrier any suitable binder(s), lubricant(s), suspending agent(s), coating agent(s), and/or solubilizing agent(s). A “pharmaceutically acceptable excipient” means an excipient that is useful in preparing a pharmaceutical composition that is generally safe, non-toxic and neither biologically nor otherwise undesirable, and includes an excipient that is acceptable for veterinary use as well as human pharmaceutical use. A “pharmaceutically acceptable excipient” as used in the present application includes both one and more than one such excipient.

It will be appreciated that pharmaceutical compositions for use in accordance with the present invention may be in the form of oral, parenteral, transdermal, inhalation, sublingual, topical, implant, nasal, or enterally administered (or other mucosally administered) suspensions, capsules or tablets, which may be formulated in conventional manner using one or more pharmaceutically acceptable carriers or excipients.

There may be different composition/formulation requirements depending on the different delivery systems. It is to be understood that not all of the compounds need to be administered by the same route. Likewise, if the composition comprises more than one active component, then those components may be administered by the same or different routes. By way of example, the pharmaceutical composition of the present invention may be formulated to be delivered using a mini-pump or by a mucosal route, for example, as a nasal spray or aerosol for inhalation or ingestible solution, or parenterally in which the composition is formulated by an injectable form, for delivery, by, for example, an intravenous, intramuscular or subcutaneous route. Alternatively, the formulation may be designed to be delivered by multiple routes.

The present invention further relates to pharmaceutical formulations containing a therapeutically effective quantity of a compound of Formula (I) or one of its salts mixed with a pharmaceutically acceptable vehicle. The pharmaceutical formulations of the present invention can be liquids that are suitable for oral, mucosal and/or parenteral administration, for example, drops, syrups, solutions, injectable solutions that are ready for use or are prepared by the dilution of a freeze-dried product but are preferably solid or semisolid as tablets, capsules, granules, powders, pellets, pessaries, suppositories, creams, salves, gels, ointments; or solutions, suspensions, emulsions, or other forms suitable for administration by the transdermal route or by inhalation.

The compounds of the invention can be administered for immediate-, delayed-, modified-, sustained-, pulsed- or controlled-release applications.

In one aspect, oral compositions are slow, delayed or positioned release (e.g., enteric especially colonic release) tablets or capsules. This release profile can be achieved without limitation by use of a coating resistant to conditions within the stomach but releasing the contents in the colon or other portion of the GI tract wherein a lesion or inflammation site has been identified. Or a delayed release can be achieved by a coating that is simply slow to disintegrate. Or the two (delayed and positioned release) profiles can be combined in a single formulation by choice of one or more appropriate coatings and other excipients. Such formulations constitute a further feature of the present invention.

Suitable compositions for delayed or positioned release and/or enteric coated oral formulations include tablet formulations film coated with materials that are water resistant, pH sensitive, digested or emulsified by intestinal juices or sloughed off at a slow but regular rate when moistened. Suitable coating materials include, but are not limited to, hydroxypropyl methylcellulose, ethyl cellulose, cellulose acetate phthalate, polyvinyl acetate phthalate, hydroxypropyl methylcellulose phthalate, polymers of metacrylic acid and its esters, and combinations thereof. Plasticizers such as, but not limited to polyethylene glycol, dibutylphthalate, triacetin and castor oil may be used. A pigment may also be used to color the film. Suppositories are be prepared by using carriers like cocoa butter, suppository bases such as Suppocire C, and Suppocire NA50 (supplied by Gattefosse Deutschland GmbH, D-Weil am Rhein, Germany) and other Suppocire type excipients obtained by interesterification of hydrogenated palm oil and palm kernel oil (C₈-C₁₈ triglycerides), esterification of glycerol and specific fatty acids, or polyglycosylated glycerides, and whitepsol (hydrogenated plant oils derivatives with additives). Enemas are formulated by using the appropriate active compound according to the present invention and solvents or excipients for suspensions. Suspensions are produced by using micronized compounds, and appropriate vehicle containing suspension stabilizing agents, thickeners and emulsifiers like carboxymethylcellulose and salts thereof, polyacrylic acid and salts thereof, carboxyvinyl polymers and salts thereof, alginic acid and salts thereof, propylene glycol alginate, chitosan, hydroxypropylcellulose, hydroxypropylmethylcellulose, hydroxyethylcellulose, ethylcellulose, methylcellulose, polyvinyl alcohol, polyvinyl pyrrolidone, N-vinylacetamide polymer, polyvinyl methacrylate, polyethylene glycol, pluronic, gelatin, methyl vinyl ether-maleic anhydride copolymer, soluble starch, pullulan and a copolymer of methyl acrylate and 2-ethylhexyl acrylate lecithin, lecithin derivatives, propylene glycol fatty acid esters, glycerin fatty acid esters, sorbitan fatty acid esters, polyoxyethylene sorbitan fatty acid esters, polyethylene glycol fatty acid esters, polyoxyethylene hydrated caster oil, polyoxyethylene alkyl ethers, and pluronic and appropriate buffer system in pH range of 6.5 to 8. The use of preservatives, masking agents is suitable. The average diameter of micronized particles can be between 1 and 20 micrometers, or can be less than 1 micrometer. Compounds can also be incorporated in the formulation by using their water-soluble salt forms.

Alternatively, materials may be incorporated into the matrix of the tablet e.g. hydroxypropyl methylcellulose, ethyl cellulose or polymers of acrylic and metacrylic acid esters. These latter materials may also be applied to tablets by compression coating.

Pharmaceutical compositions can be prepared by mixing a therapeutically effective amount of the active substance with a pharmaceutically acceptable carrier that can have different forms, depending on the way of administration. Pharmaceutical compositions can be prepared by using conventional pharmaceutical excipients and methods of preparation. The forms for oral administration can be capsules, powders or tablets where usual solid vehicles including lactose, starch, glucose, methylcellulose, magnesium stearate, di-calcium phosphate, mannitol may be added, as well as usual liquid oral excipients including, but not limited to, ethanol, glycerol, and water. All excipients may be mixed with disintegrating agents, solvents, granulating agents, moisturizers and binders. When a solid carrier is used for preparation of oral compositions (e.g., starch, sugar, kaolin, binders disintegrating agents) preparation can be in the form of powder, capsules containing granules or coated particles, tablets, hard gelatin capsules, or granules without limitation, and the amount of the solid carrier can vary (between 1 mg to 1 g). Tablets and capsules are the preferred oral composition forms.

Pharmaceutical compositions containing compounds of the present invention may be in any form suitable for the intended method of administration, including, for example, a solution, a suspension, or an emulsion. Liquid carriers are typically used in preparing solutions, suspensions, and emulsions. Liquid carriers contemplated for use in the practice of the present invention include, for example, water, saline, pharmaceutically acceptable organic solvent(s), pharmaceutically acceptable oils or fats, and the like, as well as mixtures of two or more thereof. The liquid carrier may contain other suitable pharmaceutically acceptable additives such as solubilizers, emulsifiers, nutrients, buffers, preservatives, suspending agents, thickening agents, viscosity regulators, stabilizers, and the like. Suitable organic solvents include, for example, monohydric alcohols, such as ethanol, and polyhydric alcohols, such as glycols. Suitable oils include, for example, soybean oil, coconut oil, olive oil, safflower oil, cottonseed oil, and the like. For parenteral administration, the carrier can also be an oily ester such as ethyl oleate, isopropyl myristate, and the like. Compositions of the present invention may also be in the form of microparticles, microcapsules, liposomal encapsulates, and the like, as well as combinations of any two or more thereof.

Examples of pharmaceutically acceptable disintegrants for oral compositions useful in the present invention include, but are not limited to, starch, pre-gelatinized starch, sodium starch glycolate, sodium carboxymethylcellulose, croscarmellose sodium, microcrystalline cellulose, alginates, resins, surfactants, effervescent compositions, aqueous aluminum silicates and crosslinked polyvinylpyrrolidone.

Examples of pharmaceutically acceptable binders for oral compositions useful herein include, but are not limited to, acacia; cellulose derivatives, such as methylcellulose, carboxymethylcellulose, hydroxypropyl methylcellulose, hydroxypropylcellulose or hydroxyethylcellulose; gelatin, glucose, dextrose, xylitol, polymethacrylates, polyvinylpyrrolidone, sorbitol, starch, pre-gelatinized starch, tragacanth, xanthane resin, alginates, magnesium-aluminum silicate, polyethylene glycol or bentonite.

Examples of pharmaceutically acceptable fillers for oral compositions include, but are not limited to, lactose, anhydrolactose, lactose monohydrate, sucrose, dextrose, mannitol, sorbitol, starch, cellulose (particularly microcrystalline cellulose), dihydro- or anhydro-calcium phosphate, calcium carbonate and calcium sulfate.

Examples of pharmaceutically acceptable lubricants useful in the compositions of the invention include, but are not limited to, magnesium stearate, talc, polyethylene glycol, polymers of ethylene oxide, sodium lauryl sulfate, magnesium lauryl sulfate, sodium oleate, sodium stearyl fumarate, and colloidal silicon dioxide.

Examples of suitable pharmaceutically acceptable odorants for the oral compositions include, but are not limited to, synthetic aromas and natural aromatic oils such as extracts of oils, flowers, fruits (e.g., banana, apple, sour cherry, peach) and combinations thereof, and similar aromas. Their use depends on many factors, the most important being the organoleptic acceptability for the population that will be taking the pharmaceutical compositions.

Examples of suitable pharmaceutically acceptable dyes for the oral compositions include, but are not limited to, synthetic and natural dyes such as titanium dioxide, beta-carotene and extracts of grapefruit peel.

Suitable examples of pharmaceutically acceptable sweeteners for the oral compositions include, but are not limited to, aspartame, saccharin, saccharin sodium, sodium cyclamate, xylitol, mannitol, sorbitol, lactose and sucrose.

Suitable examples of pharmaceutically acceptable buffers include, but are not limited to, citric acid, sodium citrate, sodium bicarbonate, dibasic sodium phosphate, magnesium oxide, calcium carbonate and magnesium hydroxide.

Suitable examples of pharmaceutically acceptable surfactants include, but are not limited to, sodium lauryl sulfate and polysorbates.

Suitable examples of pharmaceutically acceptable preservatives include, but are not limited to, various antibacterial and antifungal agents such as solvents, for example ethanol, propylene glycol, benzyl alcohol, chlorobutanol, quaternary ammonium salts, and parabens (such as methyl paraben, ethyl paraben, propyl paraben, etc.).

Suitable examples of pharmaceutically acceptable stabilizers and antioxidants include, but are not limited to, ethylenediaminetetriacetic acid (EDTA), thiourea, tocopherol and butyl hydroxyanisole.

The compounds of the invention may also, for example, be formulated as suppositories e.g., containing conventional suppository bases for use in human or veterinary medicine or as pessaries e.g., containing conventional pessary bases.

The compounds according to the invention may be formulated for topical administration, for use in human and veterinary medicine, in the form of ointments, creams, gels, hydrogels, lotions, solutions, shampoos, powders (including spray or dusting powders), pessaries, tampons, sprays, dips, aerosols, drops (e.g., eye ear or nose drops) or pour-ons.

For application topically to the skin, the agent of the present invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax, sorbitan monostearate, a polyethylene glycol, liquid paraffin, polysorbate 60, cetyl esters wax, cetearyl alcohol, 2-octyldodecanol, benzyl alcohol, and water. Such compositions may also contain other pharmaceutically acceptable excipients, such as polymers, oils, liquid carriers, surfactants, buffers, preservatives, stabilizers, antioxidants, moisturizers, emollients, colorants, and odorants.

Examples of pharmaceutically acceptable polymers suitable for such topical compositions include, but are not limited to, acrylic polymers; cellulose derivatives, such as carboxymethylcellulose sodium, methylcellulose or hydroxypropylcellulose; natural polymers, such as alginates, tragacanth, pectin, xanthan and cytosan.

As indicated, the compound of the present invention can be administered intranasally or by inhalation and is conveniently delivered in the form of a dry powder inhaler or an aerosol spray presentation from a pressurized container, pump, spray or nebulizer with the use of a suitable propellant, e.g., a hydrofluoroalkane such as 1,1,1,2-tetrafluoroethane (HFA 134AT) or 1,1,1,2,3,3,3-heptafluoropropane (HFA 227EA), or a mixture thereof. In the case of a pressurized aerosol, the dosage unit may be determined by providing a valve to deliver a metered amount. The pressurized container, pump, spray or nebulizer may contain a solution or suspension of the active compound, e.g., using a mixture of ethanol and the propellant as the solvent, which may additionally contain a lubricant, e.g., sorbitan trioleate.

Capsules and cartridges (made, for example, from gelatin) for use in an inhaler or insufflator may be formulated to contain a powder mix of the compound and a suitable powder base such as lactose or starch.

For topical administration by inhalation the compounds according to the invention may be delivered for use in human or veterinary medicine via a nebulizer.

The pharmaceutical compositions of the invention may contain from 0.01 to 99% weight per volume of the active material. For topical administration, for example, the composition will generally contain from 0.01-10%, more preferably 0.01-1% of the active material.

A therapeutically effective amount of the compound of the present invention can be determined by methods known in the art. The therapeutically effective quantities will depend on the age and on the general physiological condition of the subject, the route of administration and the pharmaceutical formulation used. The therapeutic doses will generally be between about 10 and 2000 mg/day and suitably between about 30 and 1500 mg/day. Other ranges may be used, including, for example, 50-500 mg/day, 50-300 mg/day, 20-200 mg/day, 100-200 mg/day. The daily dose as employed for adult human treatment will range from 0.01 to 250 mg/kg body weight, suitably 2-100 mg/kg body weight, or suitably 5-60 mg/kg body weight, which may be administered in one to four daily doses, for example, depending on the route of administration and the condition of the subject. When the composition comprises dosage units, each unit will generally contain 10 mg to 2 g of active ingredient, suitably 200 mg to 1 g of active ingredient. The amount of the compound required for prophylactic treatment, referred to as a prophylactically-effective dosage, is generally the same as described for therapeutic treatment although it may be desirable to use a smaller dose and/or less frequent dosing, such as once per week.

Administration may be once a day, twice a day, or more often, and may be decreased during a maintenance phase of the disease or disorder, e.g. once every second or third day instead of every day or twice a day. The dose and the administration frequency will depend on the clinical signs, which confirm maintenance of the remission phase, with the reduction or absence of at least one or more preferably more than one clinical signs of the acute phase known to the person skilled in the art.

### Method of Preparation:

Compounds of Formula (I) and salts thereof may be prepared by the general methods outlined hereinafter, said methods constituting a further aspect of the invention. In the following description, the groups R¹, R², R³, R⁴, R⁵, R⁶, X, A, a, b and c have the meaning defined for the compounds of Formula (I) unless otherwise stated.

It will be appreciated by those skilled in the art that it may be desirable to use protected derivatives of intermediates used in the preparation of the compounds of Formula (I). Protection and deprotection of functional groups may be performed by methods known in the art. Hydroxyl or amino groups may be protected with any hydroxyl or amino protecting group (for example, as described in Green and Wuts. Protective Groups in Organic Synthesis. John Wiley and Sons, New York, 1999). The protecting groups may be removed by conventional techniques. For example, acyl groups (such as alkanoyl, alkoxycarbonyl and aryloyl groups) may be removed by solvolysis (e.g., by hydrolysis under acidic or basic conditions). Arylmethoxycarbonyl groups (e.g., benzyloxycarbonyl) may be cleaved by hydrogenolysis in the presence of a catalyst such as palladium-on-carbon. 1,2 diol groups may be protected as acetal by reaction with dimethyl acetal of N,N-dimethylacetamide (DMADMA) or dimethyl acetal of N,N-dimethylformamide (DMFDMA) which may be removed by hydrogenolysis or methanolisis at reflux (Tetrahedron Lett. 12 (1971), 813-816, Collection Czech. Chem. Commun. 32 (1967), 3159).

The synthesis of the target compound is completed by removing any protecting groups, which are present in the penultimate intermediate using standard techniques, which are well-known to those skilled in the art. The final product is then purified, as necessary, using standard techniques such as silica gel chromatography, HPLC on silica gel, and the like or by recrystallization.

Compounds of Formula (I) wherein R¹ is a group of Formula a), X is divalent radical —N(R⁵)—, a is 2-6, b is 1-6 and c is 0, may be prepared by reaction of an amine of Formula (III) wherein R⁷ is H or hydroxyl protecting group,

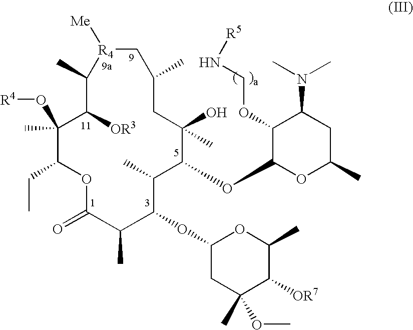

by reductive amination with a suitable aldehyde of formula (IV)

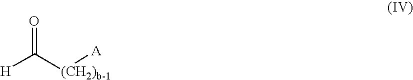

The reductive amination reaction is preferably carried out in a solvent such as methanol, DMF or a mixture thereof. A suitable reducing agent is, for example sodium cyanoborohydride.

Compounds of Formula (I) wherein R¹ is a group of Formula a), X is divalent radical —N(R⁵)—, a is 2-6, b is 0, and c is 0, may be prepared by reaction of an amine of Formula (III) with a reagent of Formula (Va)

L-A (Va)

wherein L represents leaving group. Leaving group L may be any leaving group known in the art to be suitable for this type of reaction. Preferably, L is selected from chloride, bromide, iodide, tosyloxy and methanesulfonyloxy group.

The reaction is preferably carried out in a solvent such as a halohydrocarbon (e.g. dichloromethane), an ether (e.g. tetrahydrofuran or dimethoxyethane), acetonitrile or ethyl acetate and the like, dimethylsulfoxide, N,N-dimethylformamide or 1-methyl-pyrrolidone and in the presence of a base, followed, if desired, by removal of the hydroxyl protecting groups. Examples of suitable bases include organic bases such as diisopropylethylamine, triethylamine and 1,8-diazabicyclo[5.4.0]undec-7-ene (DBU), and inorganic bases such as potassium hydroxide, cesium hydroxide, tetraalkylammonium hydroxide, sodium hydride, potassium hydride and the like.

Compounds of Formula (I) wherein R¹ is a group of Formula a), X is divalent radical —N(R⁵)—, a is 2-6, b is 1-6, and c is 1, may be prepared by reaction of an amine of Formula (III) with a reagent of Formula (Vb)

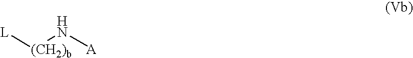

wherein L represents leaving group under reaction condition as described above for compounds of Formula (III) and (Va). Leaving group L may be any leaving group known in the art to be suitable for this type of reaction. Preferably, L is selected from chloride, bromide, iodide, tosyloxy and methanesulfonyloxy group.

Compounds of Formula (I) wherein R¹ is a group of Formula a), X is divalent radical —NHC(O)—, a is 2-6, b is 0-6, and c is 0, may be prepared by reaction of an amine of Formula (III) wherein R⁵ is H and R⁷ is H or hydroxyl protecting group, with a compound of Formula (VI)

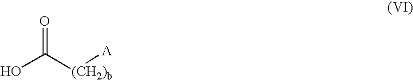

in the presence of carbodiimides such as dicyclohexylcarbodiimide (DCC), 1,8-diazabicyclo[5.4.0.]undec-7-ene (DBU) or 1-(3-dimethylaminopropyl)-3-ethylcarbodiimide (EDC) in the presence of hydroxybenzotriazole monohydrate (HOBt) in a suitable aprotic solvent such as a halohydrocarbon (e.g. dichloromethane) or N,N-dimethylformamide optionally in the presence of a tertiary organic base such as dimethylaminopyridine or triethylamine or in the presence of an inorganic base (eg. sodium hydroxide) and at a temperature within the range of 0° to 120° C.

In yet another embodiment, compounds of Formula (I), wherein R¹ is a group of Formula a), X is divalent radical —C(O)NH—, a is 2-6, b is 0-6, and c is 0, may be prepared by reaction of a compound of Formula (VII) wherein R⁷ is H or hydroxyl protecting group

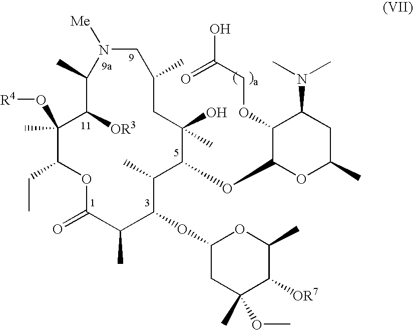

with an amine of Formula (VIII)

H₂N—(CH₂)_{b}-A (VIII).

The reaction is suitably carried out in a suitable inert solvent such as halohydrocarbon (e.g. dichloromethane) or N,N-dimethylformamide, lower alcohol (e.g. tert-butanol, iso-propanol, ethanol or methanol) optionally in the presence of EDC, an organic base such as dimethylaminopyridine, triethylamine or DBU, or an inorganic base such as sodium hydroxide, lithium hydroxide or potassium hydroxide, and at a temperature within the range from 0 to 120° C.

In a further embodiment, compounds of Formula (I) wherein R¹ is a group of Formula a), X is divalent radical —N(R⁵)—, a is 2-6, b is 1-6, and c is 1, may be prepared by reaction of a compound of Formula (III) wherein R⁷ is H or hydroxyl protecting group, with a suitable aldehyde of Formula (IX)

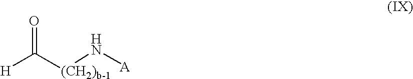

by reductive amination under conditions as described above for the reaction of compounds of Formula (III) and (IV).

In a further embodiment, compounds of Formula (I) wherein R¹ is a group of Formula a), X is divalent radical —NHC(O)—, a is 2-6, b is 1-6, and c is 1, may be prepared by reaction of a compound of Formula (III) wherein R⁵ is hydrogen, with a compound of Formula (X)

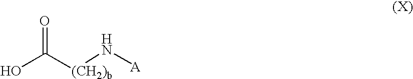

in the presence of carbodiimides such as dicyclohexylcarbodiimide (DCC), 1,8-diazabicyclo[5.4.0.]undec-7-ene (DBU) or 1-(3-dimethylaminopropyl)-3-ethylcarbodiimide (EDC), in the presence of hydroxybenzotriazole monohydrate (HOBt) in a suitable aprotic solvent such as a halohydrocarbon (e.g. dichloromethane) or N,N-dimethylformamide, optionally in the presence of a tertiary organic base such as dimethylaminopyridine or triethylamine or in the presence of an inorganic base (eg. sodium hydroxide), and at a temperature within the range of 0 to 120° C.

In yet another embodiment, compounds of Formula (I) wherein R¹ is a group of Formula a), X is divalent radical —C(O)NH—, a is 2-6, b is 1-6, and c is 1, may be prepared by reaction of a compound of Formula (VII) wherein R⁷ is H or hydroxyl protecting group, with compound of Formula (XI) NH₂—(CH₂)_{b}—NH-A (XI).

The reaction is suitably carried out in a suitable inert solvent such as a halohydrocarbon (e.g. dichloromethane) or N,N-dimethylformamide, lower alcohol (e.g. tert-butanol, iso-propanol, ethanol or methanol), optionally in the presence of EDC, an organic base such as dimethylaminopyridine, triethylamine or DBU, or an inorganic base such as sodium hydroxide, lithium hydroxide or potassium hydroxide, and at a temperature within the range of 0 to 120° C.

Compounds of Formula (III) wherein R⁵ is hydrogen and a is an integer from 2 to 6, may be prepared from compounds of Formula (XII) wherein a′ is an integer from 1 to 5

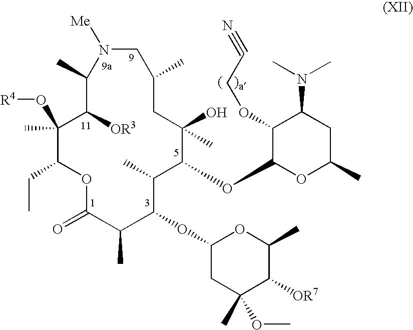

by reduction of the cyano nitrogen to —NH₂.

The reaction is suitably carried out in a suitable solvent such as acetic acid using suitable reduction conditions, such as hydrogenation in the presence of a suitable catalyst such as platinum dioxide at a suitable pressure, such as 5 barr.

Compounds of Formula (XII) wherein a′ is an integer from 2 to 6 may be prepared by reaction of a compound of Formula (XIII) wherein R³, R⁴ and R⁷ are suitable hydroxy protecting groups

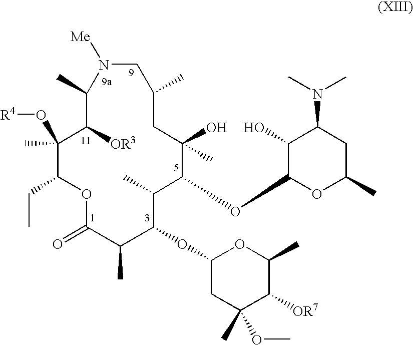

with a suitable vinyl nitrile, for example acrylonitrile in the case where a′ is 2, in the presence of a strong base, such as NaOH, KOtBu, NaOtBu or NaH, in a suitable solvent such as DMSO or t-BuOH.

Compounds of Formula (XII) wherein a′ is 1 may be prepared from compounds of Formula (XIII), wherein R³, R⁴ and R⁷ are suitable hydroxy protecting groups, by reaction with a suitable monohalogenated acetonitrile, for example chloracetonitrile, in the presence of a strong base, such as NaOH, KOtBu, NaOtBu or NaH, in a suitable solvent such as DMSO or t-BuOH.

Compounds of Formula (XII) wherein a′ is an integer of 4 or 5 may be prepared from compounds of Formula (XIV)

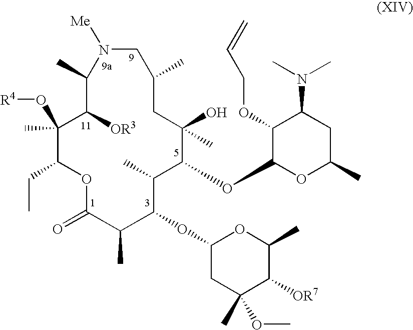

wherein R³, R⁴ and R⁷ are suitable hydroxy protecting groups, by reaction with a compound of Formula (XV)

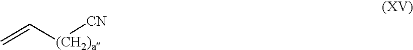

wherein a″ is an integer of 1 or 2, under conditions of Grubbs metathesis (A. K. Chatterjee, T.-L. Choi, D. P. Sanders, R. H. Grubbs, JACS 125 (2003) 11360). Selective reduction of the double bond (and not the —CN group) may be achieved by hydrogenation in the presence of Pd/C catalyst in a suitable solvent, such as an alcohol such as ethanol or methanol (J. Med. Chem. 51 (2008) 424-431).

Compounds of Formula (III) wherein a is an integer of 5 or 6 may also be prepared from compounds of Formula (XIV) and (XV) using Grubbs metathesis as described above, but using acidic reduction conditions such as hydrogenation in the presence of a suitable catalyst such as platinum dioxide at a suitable pressure, such as 5 barr, in a suitable solvent such as acetic acid.

Compounds of Formula (XIV) may be prepared by palladium-catalysed allylation of compounds of Formula (XIII), for example according to the procedure described in WO 2006/120541 for Intermediate 16.

Compounds of formula (VII) wherein R³, R⁴ and R⁷ are suitable hydroxy protecting groups and a is 2 may be prepared by reaction of a compound of Formula (XIII) with methyl acrylate in the presence of a strong base, such as NaOH, KOtBu, NaOtBu or NaH, in a suitable solvent such as DMSO or t-BuOH, followed by ester hydrolysis under conditions well known to those skilled in the art.

Compounds of Formula (VII) in which a is an integer of 2 to 6 may be prepared by hydrolysis of a compound of Formula (XII) in which a′ is an integer of 2 to 6.

Compounds of Formula (VII) in which a is an integer of 2 to 6 may be prepared by oxidation of a compound of Formula (XVI)

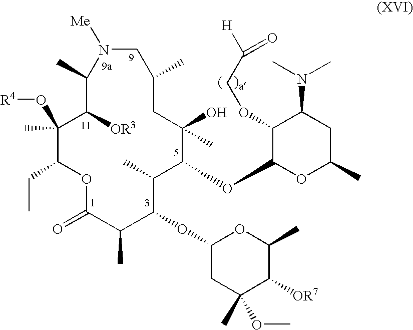

wherein a′ is an integer from 2 to 6.

Compounds of Formula (VII) in which a is 6 may be prepared from compounds of Formula (XVI) in which a′ is 4 by a Wittig reaction with (Ph)₃P═C—CO₂CH₃, followed by selective reduction of the double bond, for example by hydrogenation in the presence of Pt or Pd as catalyst, and ester hydrolysis under basic conditions.

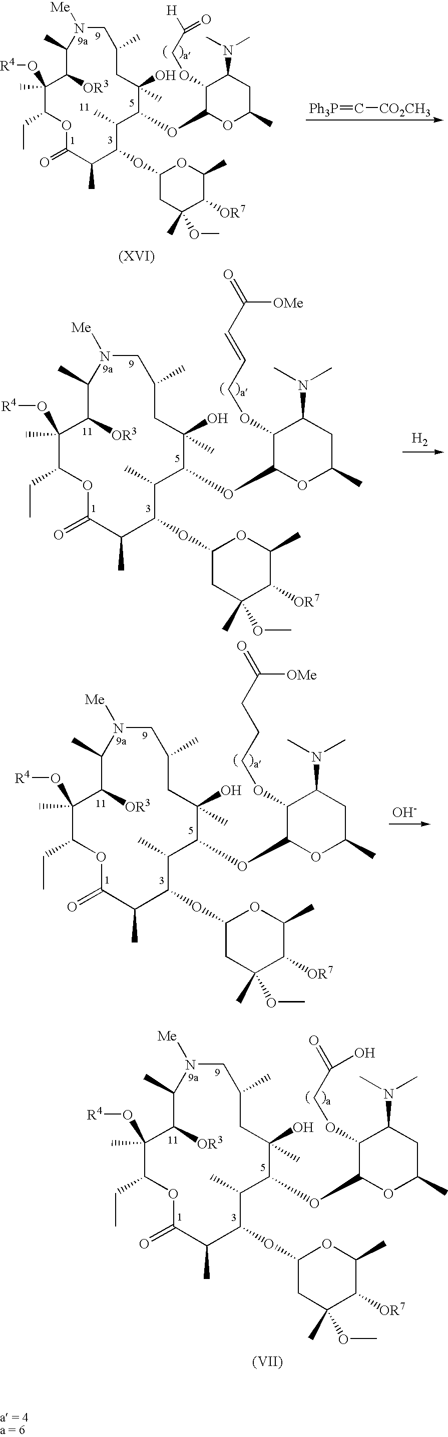

Compounds of Formula (VII) in which a is an integer from 4 to 6 may be prepared by reduction of a compound of Formula (XVI) in which a′ is an integer from 3 to 5, for example using sodium borohydride to yield an alcohol intermediate, followed by halogenation, for example using SOCl₂, followed by Grignard reaction, for example using magnesium in the presence of CO₂.

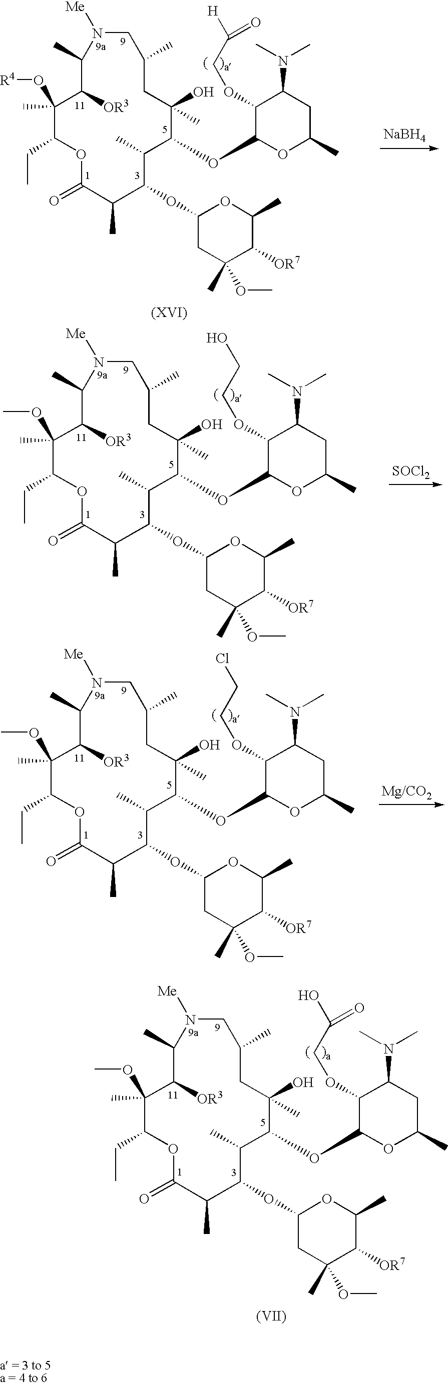

Compounds of Formula (XVI) wherein a′ is 2 to 6, may be prepared from compounds of Formula (VII) wherein a is 2 to 6, by reduction with DIBAI-H (diisobutylaluminium hydride) in dry THF at a low temperature, suitably around −78° C.

Compounds of Formula (XVI) wherein a′ is 2 or 3, may be prepared from compounds of Formula (XIV) wherein R³, R⁴ and R⁷ are suitable hydroxy protecting groups, by hydroboration with 9-BBN, or other suitable boranes, followed by treatment with peroxide and then oxidation (a=3) or by osmium tetroxide/periodate cleavage (a=2) according to the procedure described in WO 2006/120541.

Compounds of Formula (I) wherein R¹ is hydrogen may be prepared by acid hydrolysis with diluted hydrochloric acid of compound of Formula (I) wherein R¹ is a group of Formula (a) at a temperature within the range of 20° to 40° C.

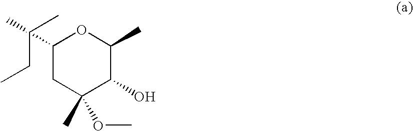

Compounds of Formula (I) wherein R³ and R⁴ taken together with the intervening atoms form a cyclic carbonate group of Formula (b);

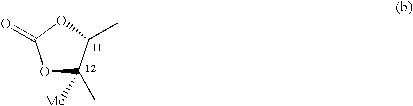

may be prepared by analogous methods to those known in the art from compounds of Formula (I) wherein R³ and R⁴ are H. Thus, they can be prepared according to the procedure in J. Antibiot. 40 (1987), 1006-1015 and EP0307177.

Compounds of Formula (I) wherein R⁵ is C₁₋₃alkyl may be prepared by alkylation of compounds of Formula (I) wherein R⁵ is hydrogen, for example where R⁵ is methyl by alkylating a chloroform solution of the compound wherein R⁵ is hydrogen with formaldehyde in the presence of formic acid.

Compounds of Formula (I) wherein A is a compound of Formula c) or d) and R⁶ is hydrogen may be prepared by hydrogenation of the corresponding compound of Formula (I) wherein R⁶ is chlorine, for example by exposure to a hydrogen atmosphere in the presence of 10% Pd/C catalyst.

Compounds of Formulae IV, Va, Vb, VI, VIII, IX, X, XI and XIII are commercially available or may be readily prepared by methods well known in the art.

Compounds of Formula (II), which are a subset of compounds of Formula (III), may be prepared in a similar manner to that described for compounds of Formula (III) by selection of the appropriate starting materials.

Compounds of Formula (II) may also be prepared by hydrolysis in basic conditions of a compound of Formula (III) in which R³ and R⁴ taken together with the intervening atoms form a cyclic group of Formula (b).

Salts, such as pharmaceutically acceptable acid addition salts, which also represent an object of the present invention, may be obtained by reaction of a compound of Formula (I) with an at least equimolar amount of the corresponding inorganic or organic acid such as hydrochloric acid, hydroiodic acid, sulfuric acid, phosphoric acid, acetic acid, trifluoroacetic acid, propionic acid, benzoic acid, benzenesulfonic acid, methane sulfonic acid, laurylsulfonic acid, stearic acid, palmitic acid, succinic acid, ethylsuccinic acid, lactobionic acid, oxalic acid, salicylic acid and similar acid, in a solvent inert to the reaction. Addition salts are isolated by evaporating the solvent or, alternatively, by filtration after a spontaneous precipitation or a precipitation by the addition of a non-polar cosolvent.

Compounds of Formula (I) and pharmaceutically acceptable addition salts with inorganic or organic acids thereof possess an antimalarial activity in vitro.

### Biological Assays

The potential for the compounds of the present invention to have a therapeutic benefit in the treatment and/or prophylaxis of malaria may be demonstrated, for example, using the following assay:

### In Vitro Screening Protocol

### I. Materials

### Parasite.

Plasmodium falciparum strains 3D7A and W2.

### Culture Medium.

The culture medium comprised RPMI 1640 with 25 mM HEPES, sodium bicarbonate and glutamine (GIBCO™ cat. ref.: 52400), supplemented with 10% of pooled human sera AB (Bioreclamation HMSRM-AB)) and HT supplement (0.15 mM hypoxanthine and 24 μM thymidine), (GIBCO™ cat. ref.: 41065). Human sera were decomplemented 30 min. at 56° C., aliquoted and stored frozen at −20° C. until use in this culture medium.

This culture medium (“complete medium”) was usually prepared fresh just before use and pre-warmed to 37° C.

### Red Blood Cells.

Red blood cells AB—stock suspensions were prepared from whole blood bags coming from incomplete blood donation, provided by the Spanish Red Cross (<25 days after sampling). This “whole blood” was aliquoted and stored at 4° C.

To prepare red blood cells for the assay, the whole blood was centrifuged and washed 3 times with RPMI without serum. The upper phase, containing white blood cells was removed. The washed red blood cells were kept as a 50% suspension in complete medium. The prepared cells were stored at 4° C. and were employed in the assay at any time up to 4 days after preparation.

### II. Compounds.

### Compound Preparation

Test compounds were dissolved at 2 mg/ml in 100% DMSO on the day of the assay. If necessary, complete dissolution was achieved by gentle heating (the mixture was heated at a temperature <37° C.) and sonication (sonication bath).

Before test compounds were added to the parasites, the percentage of DMSO in the compound solution was reduced by further dilutions of the solution with culture medium prepared in the same way as described above for complete medium, but which did not contain hypoxanthine. The final concentration of DMSO in the assay plates was not permitted to exceed 0.2%, so that it did not produce any detectable undesired effects on the development of the parasite. For IC₅₀ determinations, 10 serial 2-fold dilutions were prepared in complete medium in the presence of a constant amount of DMSO. Any obvious signs of insolubility of the stock solutions in 100% DMSO or precipitation when these solutions were diluted in assay media, were recorded.

III. Plasmodium falciparum Culture (Parasite)

Plasmodium falciparum strains were maintained in complete medium at an hematocrit of 5% in continuous culture using a method adapted from Trager and Jensen (1, 2).

The parasitemia was calculated by counting the percentage of parasitized erythrocytes by optical microscopy. Thin films of blood were made every day from each culture flask, fixed with methanol and stained for 10 min. in Giemsa (Merck, cat. ref.: 1.09204) at 10% in buffered water pH 7.2. The glass slides were observed and counted with an optical microscope (Nikon, Eclipse E200) equipped with a 100× immersion oil objective.

The culture was maintained at an hematocrit of 5%, with a daily change of medium and was diluted when parasitemia had reached about 5%. The parasite population was asynchronous, composed of a stable proportion (≅70%) of young trophozoites (ring forms) and showed a regular rate of growth of 3 to 3.5 times the initial number of parasites daily.

Growth was achieved in culture flasks (canted neck, Corning) incubated at 37° C. under low oxygen atmosphere (5% CO₂, 5% O₂, 95% N₂).

### IV. IC₅₀ Assay

[³H] Hypoxanthine incorporation assay was conducted using a method adapted from Desjardins et al. (3). The assays were performed in 96 wells flat bottom microplates.

1. Serial dilutions of the test compounds (50 μl of a 5× solution/well) were deposited in duplicate. Compounds of this invention (Table 1) were tested in this assay. Chloroquine and Azithromycin were used as control compounds for each assay.

2. The inoculum was prepared as a suspension of parasitized red blood cells (PRBCs) at 2.5% of hematocrit and 0.5% of parasitemia in culture medium prepared in the same way as described above for complete medium, but which did not contain hypoxanthine.

3. [³H]-Hypoxanthine (Amersham Biosciences, cat. ref.: TRK74) was added extemporaneously to the inoculum suspension at a concentration of 1 μCi/ml (equating to 0.25 μCi/well). 200 μl of the resulting suspension was distributed into each well (other than the control well H12 described below) leading to a final volume of 250 μl per well, at 1% of hematocrit and 0.4% of parasitemia/well.

4. In each plate, 2 columns were reserved for control wells:
Column 11: Positive control wells: PRBCs with 0.2% DMSO—(i) to determine DMSO solvent effect on parasite growth (at a final concentration of 0.2%) and (ii) to compare with cultures treated with test compounds.
Column 12 (comprising wells A12-H12):
A12-D12—Background value wells: Uninfected RBCs—blank control to obtain the background reading from RBCs without parasites.

E12-G12—Solvent effect wells: PRBCs without DMSO—to determine DMSO solvent effect on PRBCs by comparing these wells with column 11 wells.

H12—Non-radioactive well: PRBCs with cold hypoxanthine—(i) to carry out a thin blood film to determine parasitemia value after incubation by microscopy and (ii) to ensure that the parasites have grown properly during the assay. (200 μl of inoculum suspension was prepared as described above (Items 2 and 3) but with non-tritiated hypoxanthine instead of [³H]-hypoxanthine, then added to this well to a final volume of 250 μl).

5. The plates were incubated for 48 hours at 37° C. under low oxygen atmosphere. At the end of the assay, a thin film was made with the non-radioactive sample (well H12) for a visual control of the development of the parasites. Incorporation was stopped by freezing the plates overnight at −80° C.

6. The growth was quantified by measuring the level of incorporation of [³H]-hypoxanthine into the nucleic acids of the parasite. After thawing the plates, the content of the wells was harvested on glass fibre filters (Wallac, cat. ref.: 1450-421) with a semi-automated cell-harvester (Harvester 96, TOMTEC). The filters were dried and treated with a Melt-on scintillator (Meltilex® A, PerkinElmer cat. ref.: 1450-441). Incorporation of radioactivity was measured with a α-counter (Wallac Microbeta, PerkinElmer).

The assays were repeated at least three independent times.

### V. Analysis of the Data

The value of each well was corrected by subtracting the background value from the absolute value. Background was calculated for each plate as the average value in counts per minute (cpm) of the uninfected control wells.

For each concentration of each test compound, the percentage of inhibition was then calculated by comparison with the value obtained from a control wells (average value of cpm from wells located in column 11) containing untreated PRBCs.

For each compound, non-linear regression fit (sigmoid dose-response curve) using GaphPad Prism 4.0 software is adjusted to obtain an IC₅₀ value, corresponding to the concentration which inhibits 50% of parasite development.

Results were expressed as the average IC₅₀ value±standard deviation of at least 3 independent experiments performed on different days.

### EXAMPLES

The following abbreviations are used in the text: HPLC for high performance liquid chromatography, DCM for dichloromethane, DMSO for dimethyl sulfoxide, EtOAc for ethyl acetate, MeOH for methanol, t-BuOH for tert-buthanol and THF for tetrahydrofuran, Et₃N for triethylamine, HOBT for 1-hydroxy benzotriazole hydrate, HOAc for acetic acid, Ac₂O for acetic anhydride, DCC for dicyclohexylcarbodiimide, DBU for 1,8-diazabicyclo[5.4.0.]undec-7-ene, EDC for 1-(3-dimethylaminopropyl)-3-ethylcarbodiimide, DMAP for 4-(dimethylamino)-pyridine, DMA/DMA for N,N-dimethylacetamide-dimethylacetal, DMF/DMA for N,N-dimethylformamide-dimethylacetal, DIPEA for N,N-diisoprpyethylamine, PtO₂ for platinum dioxide and r.t. for room temperature.

The compounds and processes of the present invention will be better understood in connection with the following examples, which are intended as an illustration only and not limiting the scope of the invention. Various changes and modifications to the disclosed embodiments will be apparent to those skilled in the art and such changes and modifications including, without limitation, those relating to the chemical structures, substituents, derivatives, formulations and/or methods of the invention may be made without departing from the spirit of the invention and the scope of the appended claims.

Where reactions are described as having been carried out in a similar manner to earlier, more completely described reactions, the general reaction conditions used were essentially the same. Work up conditions used were of the types standard in the art, but may have been adapted from one reaction to another. In the procedures that follow, reference to the product of a Description or Example by number is typically provided. This is provided merely for assistance to the skilled chemist to identify the starting material used. The starting material may not necessarily have been prepared from the batch referred to. All reactions were either carried out under nitrogen or may be carried out under nitrogen, unless otherwise stated.

9a-methyl-9a-aza-9-deoxo-9a-homoerythromycin A, may be prepared by the procedure as described in J. Chem. Res. (S) 1988, p 152.

### Intermediates:

### Intermediate 1

### 2′-O-(3-Aminopropyl)-9-deoxo-9a-methyl-9a-aza-9a-homoerythromycin A

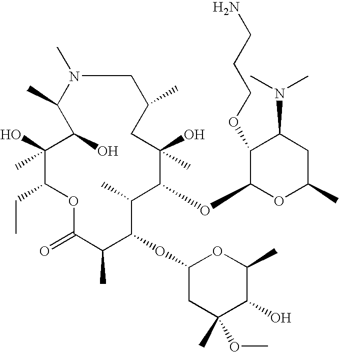

### Procedure A

### Step 1: Preparation of 3

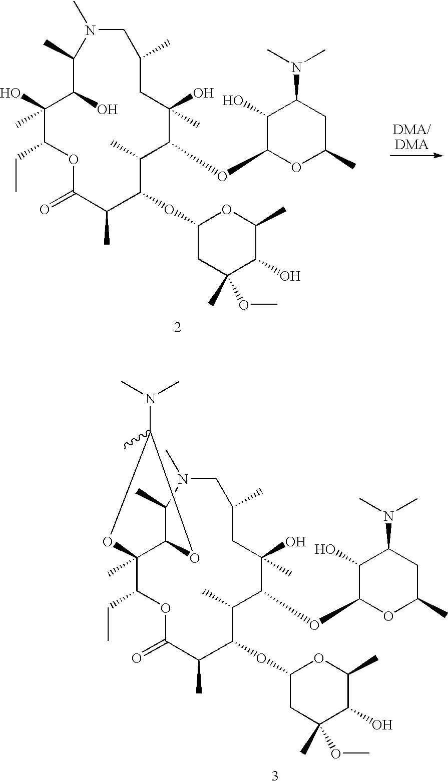

9-Deoxo-9a-methyl-9a-aza-9a-homoerythromycin A (2) (50 g, 66.7 mmol) was dissolved in chloroform (250 mL). The DMA/DMA (40 mL, 0.35 mol, 5.2 eq.) was added in one portion, and then heated at reflux temperature for 24 hours. The solvent was evaporated affording 45.28 g of the title product.

MS (ES+) m/z: 818 [MH]+.

### Step 2: Preparation of 4

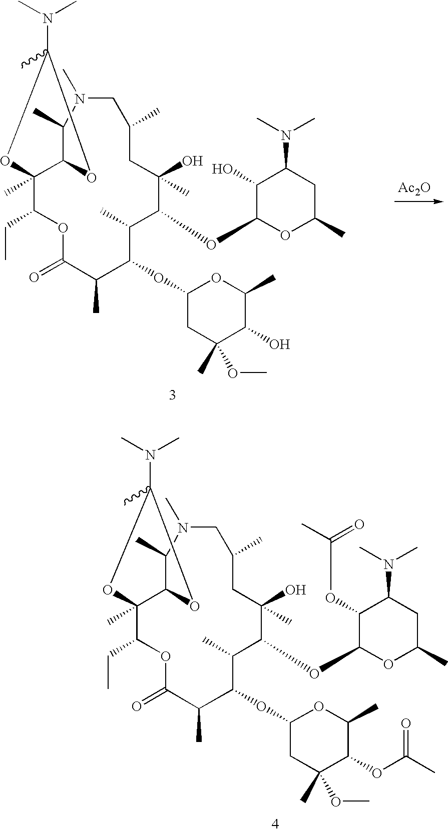

Compound 3 from step 1 (3 g, 3.66 mmol) was dissolved in DCM (70 mL) and cooled in the ice bath. In the reaction mixture Et₃N (3.2 mL, 6.2 eq.), DMAP (44.7 mg, 0.1 eq.) and Ac₂O (1.9 mL, 5.4 eq.) were added. Temperature was allowed to slowly reach r.t. and the reaction mixture was stirred at r.t. for 28 hours. Reaction mixture was washed with saturated NaHCO₃ solution (100 mL) (pH 9). DCM layers were dried over Na₂SO₄ and concentrated under vacuum affording 2.78 g of the title product. Crude product was recrystallised from Et₂O and further from acetonitrile/H₂O yielding 2.13 g of the title product as a white powder.

MS (ES+) m/z: 902.17 [MH]+

### Step 3: Preparation of 5a and 5b

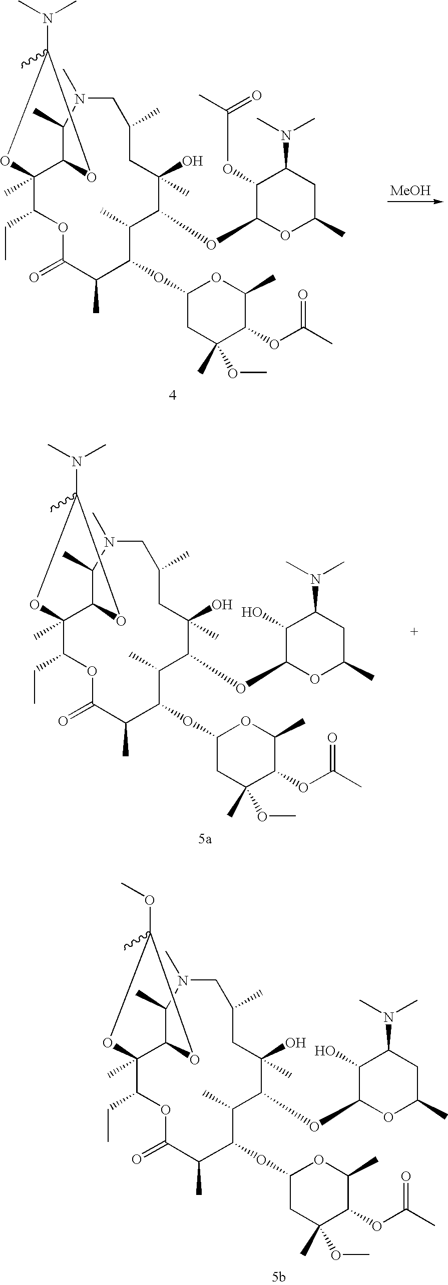

Compound 4 from step 2 (2.13 g, 2.36 mmol) was dissolved in MeOH (50 mL) and stirred at r.t. for 21 hours. Methanol was evaporated under vacuum to afford 2.08 g of the title product as white solid.

MS (ES+) m/z: 860 [MH]+ 5a
847 [MH]+ 5b

### Step 4: Preparation of 6a and 6b

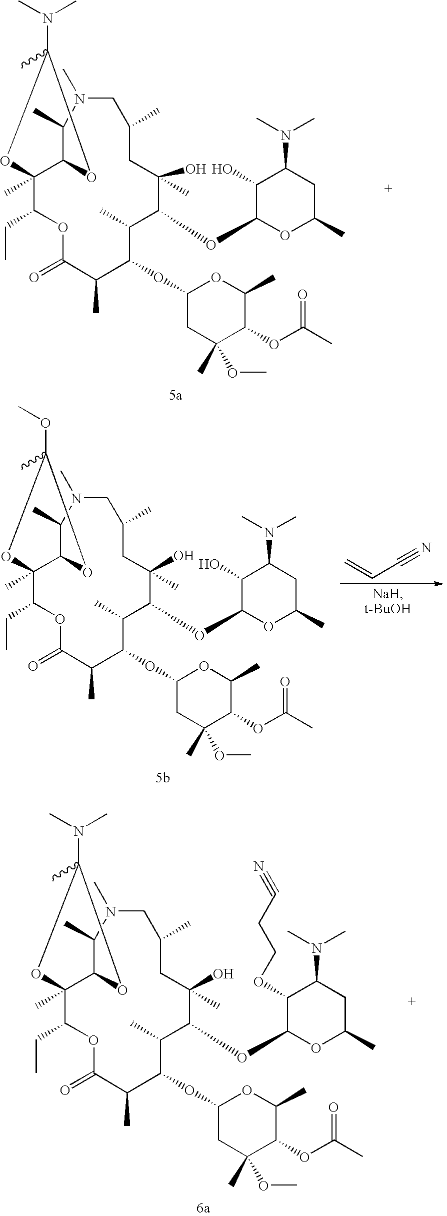

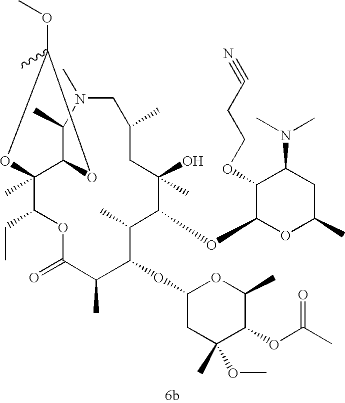

To a stirred mixture of 5a and 5b from step 3 (1.94 g, 2.25 mmol) in acrylonitrile (12 mL), t-BuOH (0.94 mL, 4.4 eq) was added at r.t. under nitrogen and the reaction mixture was cooled to 0° C. NaH (60% in mineral oil, 60 mg, 2.47 mmol, 1.1 eq.) was added in small amounts during 15 minutes. Temperature was allowed to slowly reach r.t. After 24 hours of stirring acrylonitrile was evaporated under reduced pressure. The polymer of acrylonitrile was precipitated in EtOAc/n-hexane and filtered off. The mother liquor was evaporated to afford oily product, which was then dissolved in EtOAc and extracted with water. EtOAc layers were collected and dried over Na₂CO₃. The solvent was evaporated under vacuum affording 1 g of title product.

MS (ES+) m/z: 914.10 [MH]+ 6a

MS (ES+) m/z: 900.15 [MH]+ 6b

### Step 5: Preparation of 7

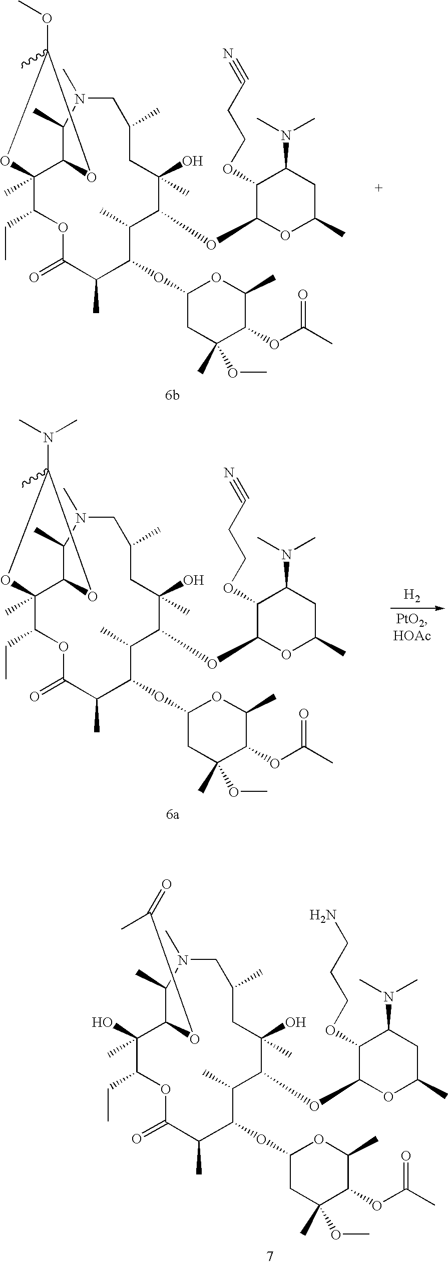

Compound 6a and 6b from step 4 (1 g), used without purification, was dissolved in glacial HOAc (10 mL) and hydrogenated with PtO₂ (100 mg) at 5 barr of H₂-pressure for 24 hours at r.t. The catalyst was filtered off and mother liquor evaporated under reduced pressure. The residue was dissolved in water and DCM, pH adjusted to 9, and extracted with DCM (150 mL). DCM layers were collected and dried over Na₂SO₄. The solvent was evaporated under reduced pressure affording 560 mg of white powder. Crude product was recrystallised from EtAc/n-hexane yielding 486.4 mg of the title product.

MS (ES+) m/z: 890.5 [MH]⁺

### Step 6: Preparation of Intermediate 1

### 2′-O-(3-Aminopropyl)-9-deoxo-9a-methyl-9a-aza-9a-homoerythromycin A

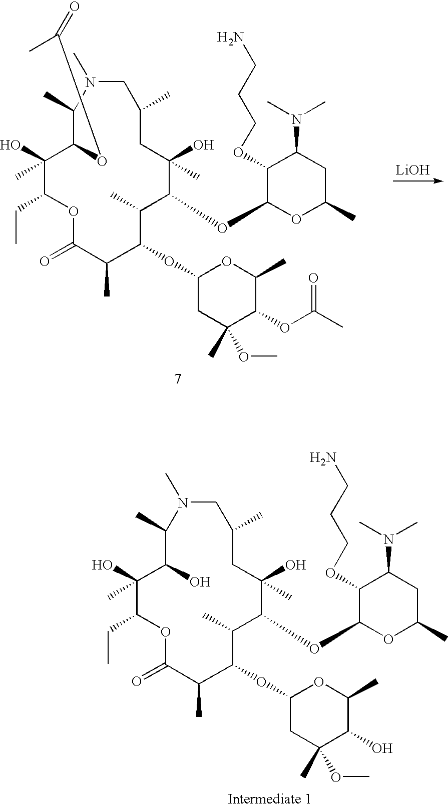

Compound 7 from step 5 (486 mg, 0.54 mmol) was dissolved in THF (4.5 mL), LiOH (6 mL of 0.5 M) was added, heated at 40° C. for 2 hours and stirred at r.t. for 72 hours. H₂O (10 mL) was added to the reaction mixture, followed by extraction with EtOAc. Organic layers were collected and dried over Na₂SO₄. Solvent was evaporated yielding 380 mg of the title product as a white solid.

MS (ES+): 806 [MH]⁺

¹³C-NMR (CDCl₃) δ/ppm: 178.9, 102.6, 94.4, 82.4, 80.6, 78.3, 77.3, 77.1, 74.1, 73.7, 73.4, 72.9, 72.6, 70.0, 68.3, 65.4, 64.4, 62.9, 49.8, 45.6, 42.5, 39.7, 39.6, 36.3, 34.6, 29.0, 27.4, 26.6, 26.5, 22.3, 21.6, 21.5, 18.4, 16.4, 14.5, 11.3, 8.5, 7.6.

### Intermediate 1

### Procedure B

### Step 1: Preparation of 8

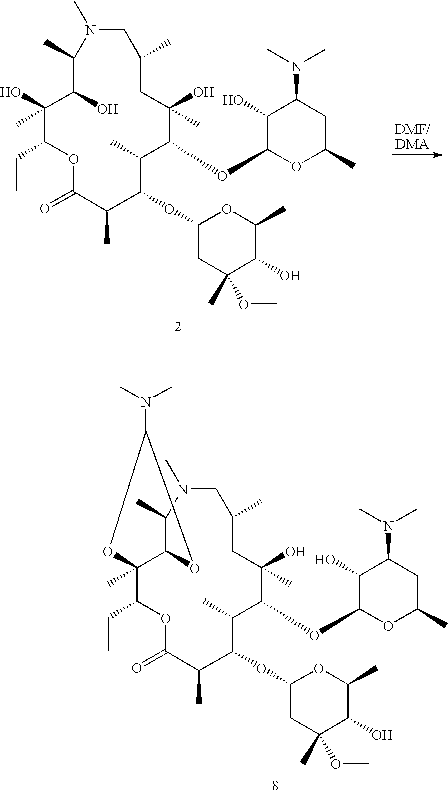

To a solution of 9-deoxo-9a-methyl-9a-aza-9a-homoerythromycin A (2) (20 g, 0.027 mol) in CHCl₃ (75 mL) DMF/DMA was added (12.5 mL, 0.093 mol), reaction was stirred at 65° C. for 5 hours and then placed at r.t. for the next 17 hours. After that time, additional DMF/DMA (12.5 mL) was added and the reaction mixture was stirred for a further 5 hours at r.t. After completion of the reaction, solvent was evaporated yielding 26.58 g of the title product as light yellow amorphous solid.

MS (ES+): 804.6 [MH]+

### Step 2: Preparation of 9

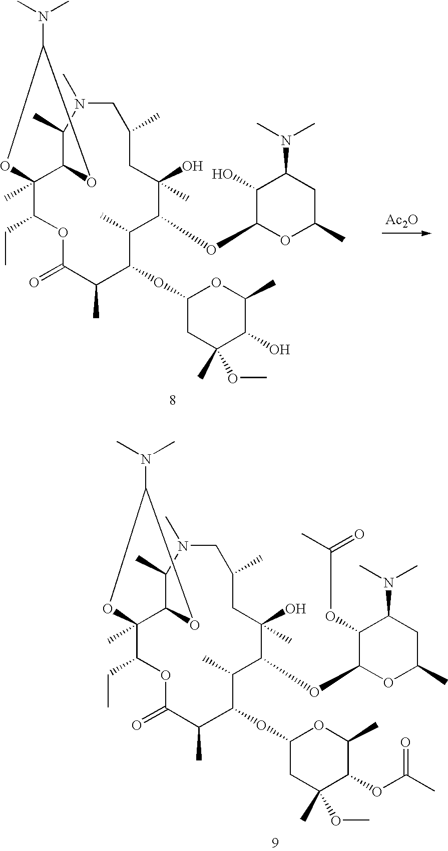

To a solution of compound 8 from step 1 (26.5 g, 0.033 mol, used without purification) in DCM (250 mL), triethylamine (28.6 mL, 0.2 mol) and DMAP (0.403 g, 0.0033 mol) were added. Solution was then cooled at 0° C. and acetic acid anhydride was added dropwise (17.1 mL, 0.18 mol). The reaction mixture stirred at r.t. for 4 hours. The organic layer was washed twice with saturated solution of NaHCO₃ then with water and brine. After drying over Na₂SO₄ and evaporation of solvent, crude product was recrystallised from diethylether to afford 8.3 g of the title product as a white crystals. After evaporating of mother liquor an additional 20.1 g of the title product was isolated.

(MS (ES+): 888.6 [MH]⁺;

### Step 3: Preparation of 10b

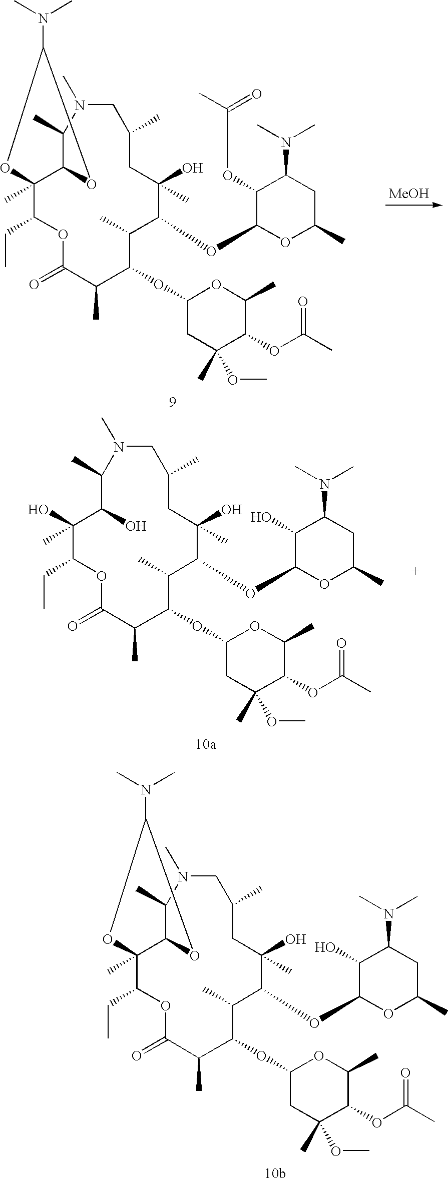

Compound 9 from step 2 (3.1 g, 3.49 mmol) was dissolved in MeOH (125 mL) and stirred at r.t. for 72 hours. Solvent was evaporated affording 2.55 g of the mixture 10a and 10b as a brown powder.

MS (ES+): 791 [MH]⁺; 10a
846 [MH]⁺ 10b

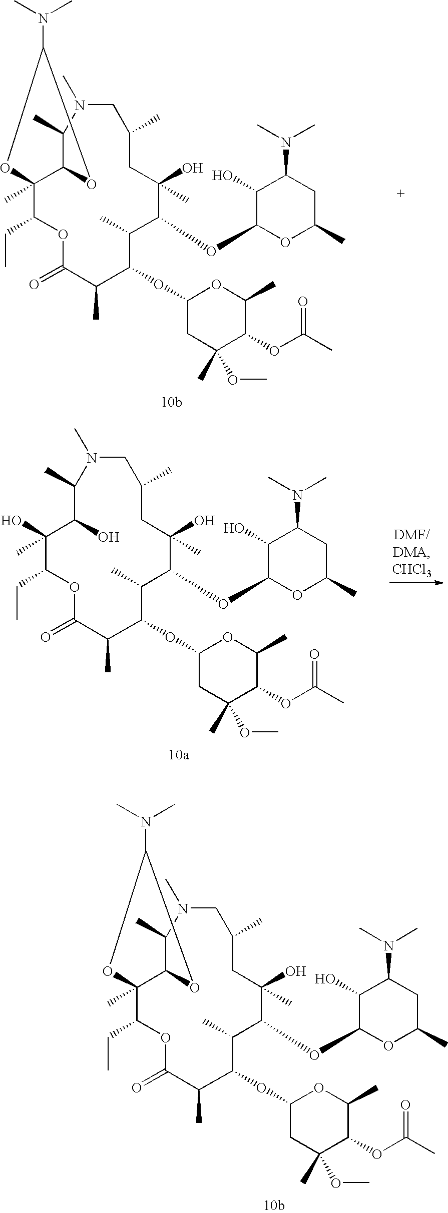

To the mixture of unprotected 10a and protected compound 10b (2.49 g) in CHCl₃ (15 mL) DMF/DMA (3 mL) was added and reaction mixture was stirred at 65° C. for 3 hours. Reaction mixture was cooled at r.t., solvent evaporated affording 3 g of the title product as a light yellow solid.

MS (ES+): 846 [MH]⁺

### Step 4: Preparation of 11a and 11b

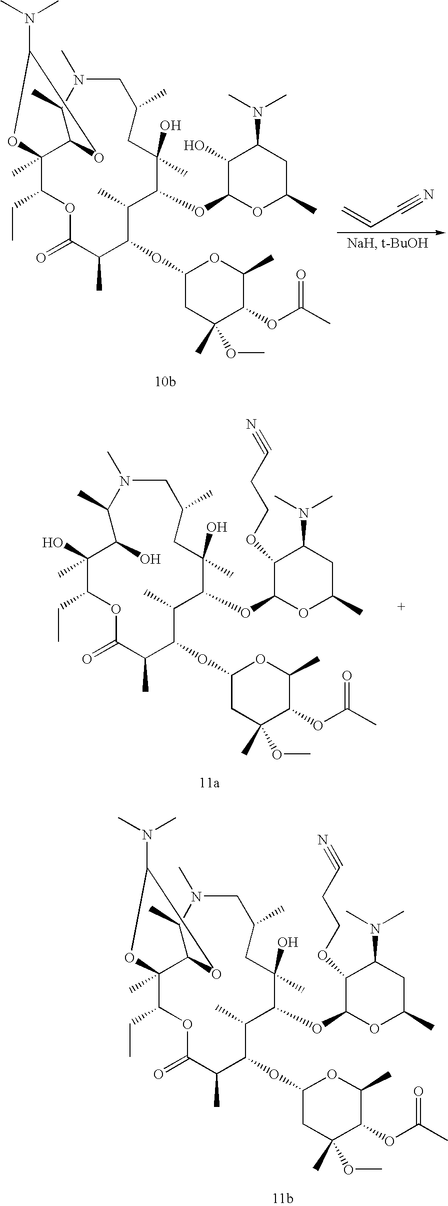

Compound 10b from step 3 (2.56 g, 3.03 mmol) was dissolved in acrylonitrile (25 mL), t-BuOH (1.5 mL, 15.9 mmol) was added and the reaction mixture was cooled at 0° C., followed by addition of NaH (112 mg, 3.33 mmol, 60% suspension in mineral oil). Reaction mixture was stirred for 3 hours and then evaporated. The residue was dissolved in EtOAc and washed with water and brine. Organic layers were dried over Na₂SO₄ and solvent was evaporated yielding 2.4 g of brown oil product which was further purified by column chromatography (DCM:MeOH:NH₄OH 90:9:0.5) yielding 1.69 g of title products.

MS (ES+): 844 [MH]⁺ 11a
899 [MH]⁺ 11b

### Step 5: Preparation of 12

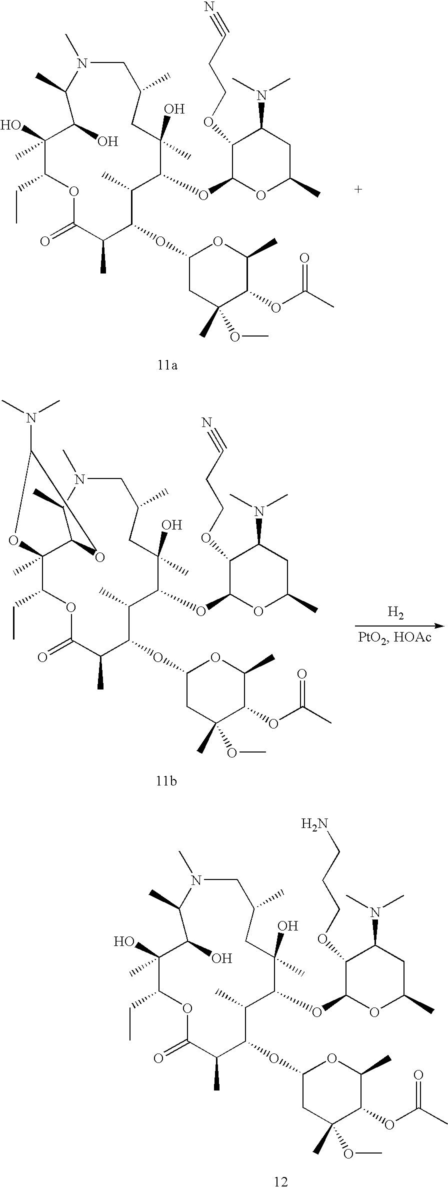

To a solution of 11a and 11b from step 4 (1.69 g) in HOAc (30 mL), PtO₂ (301 mg) was added and the reaction mixture was stirred at r.t. under 5 barr of H₂-pressure for 20 hours. The catalyst was filtered off, solvent was evaporated and residue dissolved in water and DCM. pH value was adjusted to 9.3 by addition of 1M NaOH and product was extracted with DCM. Collected organic layers were dried over Na₂SO₄ and solvent was evaporated. Crude product was recrystallised from EtOAc/n-hexane yielding 1.23 g of the title product as a white powder.

MS (ES+): 848.5 [MH]⁺

### Step 6: Preparation of Intermediate 1

### 2′-O-(3-Aminopropyl)-9-deoxo-9a-methyl-9a-aza-9a-homoerythromycin A

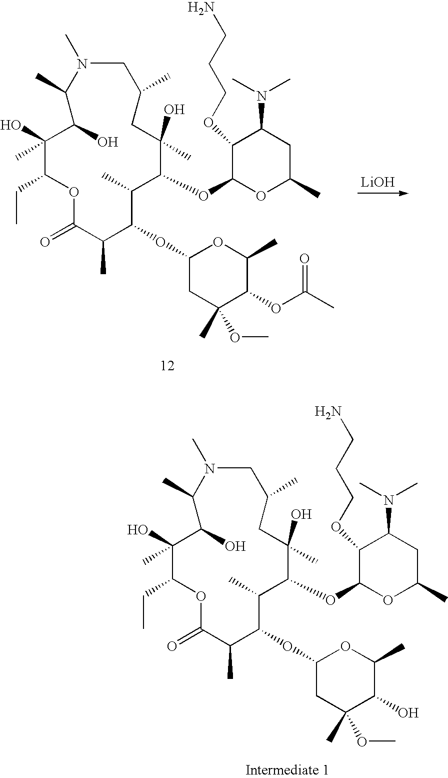

To a solution of 12 from step 5 (1.67 g, 1.97 mmol) in THF (15 mL), LiOH (10 mL of 0.5M solution in H₂O) was added. The reaction mixture was stirred for 5 hours at 65° C. and additionally 72 hours at r.t. The reaction mixture was diluted with EtOAc and washed with water. Organic layers were dried over Na₂SO₄ and solvent was evaporated yielding 1.3 g of white powder which was further purified by column chromatography (DCM:MeOH:NH₄OH 90:9:0.9) yielding 0.77 g of the title product as a white powder.

MS (ES+): 806.3 [MH]⁺

### Intermediate 2

### 11-O-Acetyl-2′-O, 4″-O-di-(3-aminopropyl)-9-deoxo-9a-methyl-9a-aza-9a-homo-erythromycin A

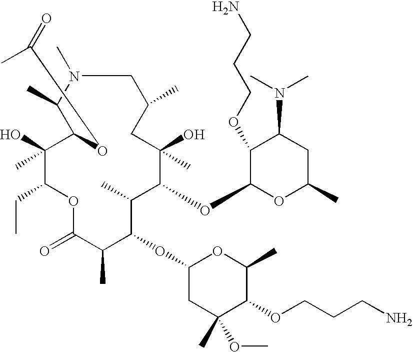

### Step 1: Preparation of 13

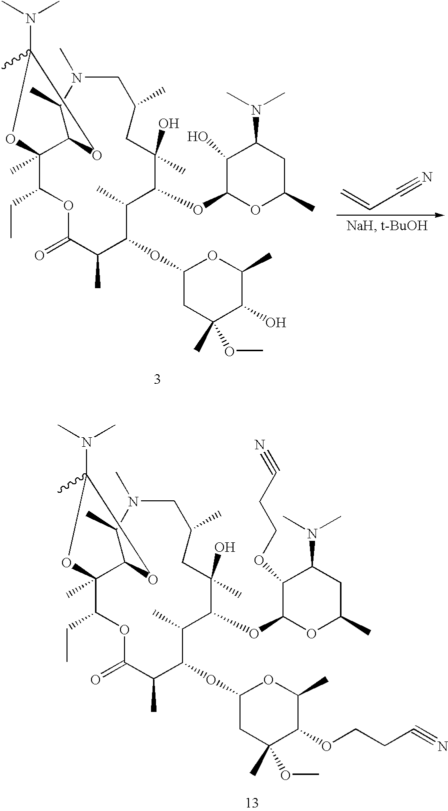

Compound 3 from step 1 for Intermediate 1 (10 g, 12.22 mmol) was dissolved in acrylonitrile (68.06 mL, 1.04 mol), t-BuOH (3.5 mL) was added, the reaction mixture cooled at 0° C., NaH (538 mg, 15.44 mmol, 60% suspension in mineral oil) was added and the reaction mixture was stirred at 4° C. for 20 hours. Precipitate was filtered off and mother liquor evaporated. The residue was dissolved in EtOAc, washed with saturated NaHCO₃ and organic extracts were dried over Na₂SO₄. Solvent was evaporated and crude product crystallised from DCM/n-hexane yielding 6.4 g of the title product.

MS (ES+) m/z: 924 [MH]+

### Step 2: Preparation of Intermediate 2

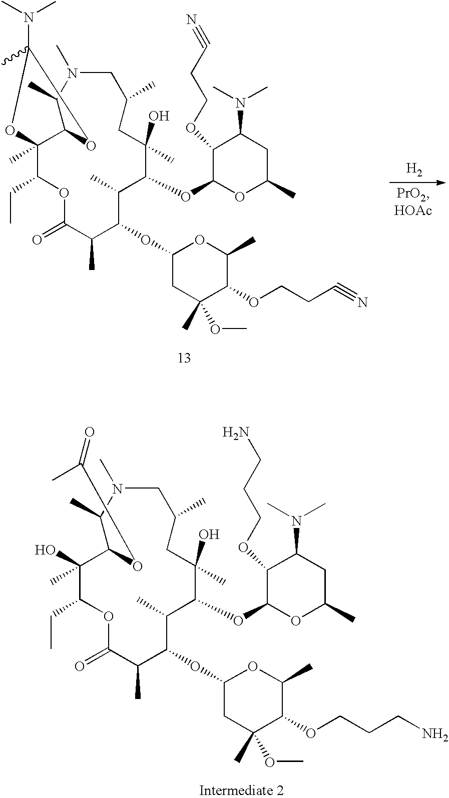

Compound 13 from step 1 (6.3 g, 7.2 mmol) was dissolved in glacial HOAc (115 mL) and hydrogenated at 5 barr of H₂-pressure in the presence of PtO₂ (630 mg) for 24 hours at r.t. The catalyst was filtered off and mother liquor evaporated under reduced pressure. To the residue, water (50 mL) and DCM (100 mL) were added, pH adjusted to 8 and extracted with DCM. Organic extracts were collected and dried over Na₂SO₄. The solvent was evaporated under reduced pressure affording 5.60 g of white powder. Crude product was recrystallised from DCM/ether yielding 4.68 g of the title product.

MS (ES+) m/z: 905.5-[MH]⁺

### Intermediate 3

### N-(7-chloro-4-quinolinyl)-1,2-ethanediamine

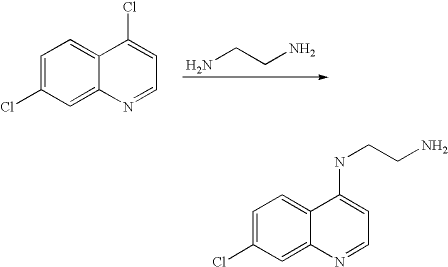

Ethylenediamine (1.7 mL, 26.5 mmol) was added to crude 4,7-dichloroquinoline (1.052 g, 5.31 mmol) and the mixture heated at 80° C. for 1 h without stirring and then 3 h at 135° C. with stirring. Reaction mixture was cooled to r.t. and 10% NaOH (30 mL) was added (pH=14) and extracted with hot EtOAc (3×50 ml). Organic layers were collected and dried on Na₂SO₄. EtOAc was evaporated under reduced pressure to afford yellow solid which was further recrystallised from hot Ethyl Acetate (150 ml). Yellow crystals were formed by cooling and were filtrated off (0.45 g). Mother liquor was evaporated under reduced pressure to afford crude material (0.49 g) which was further recrystallised as described above to yield the title product (0.347 g) as yellow crystals.

MS (ES+): 222 [MH]⁺

¹³C-NMR (DMSO) δ/ppm: 159.5, 152.3, 150.6, 150.5, 149.4, 133.7, 127.8, 124.5, 124.3, 124.2, 117.8 99.0, 98.8, 45.5, 43.9.

### Intermediate 4

### 2′-O-(3-Carboxyethyl)-9-deoxo-9a-methyl-9a-aza-9a-homoerythromycin A

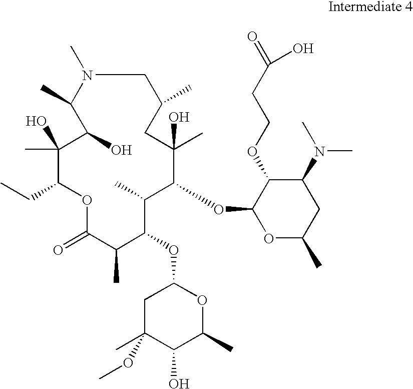

### Step 1: Preparation of 14

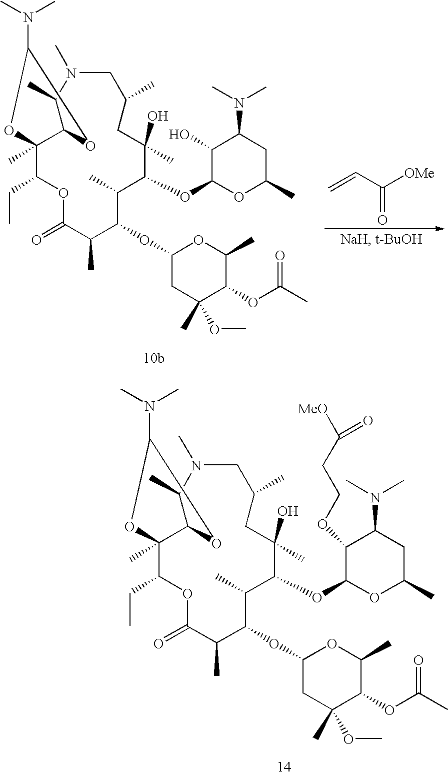

Compound 10b (Intermediate 1, step 3; 2 g, 2.36 mmol), t-BuOH (0.7 mL, 7.4 mmol) and NaH (0.2 g, 60% suspension in mineral oil) were added to methylacrylate (15 mL) cooled at 0-5° C. The reaction mixture was stirred for 3 hours and further NaH was added (0.1 g). After 20 h of stirring methylacrylate was evaporated and crude product (2.8 g) was used in the next reaction step without purification.

MS (ES+): 932.6 [MH]⁺

### Step 2: Preparation of 15

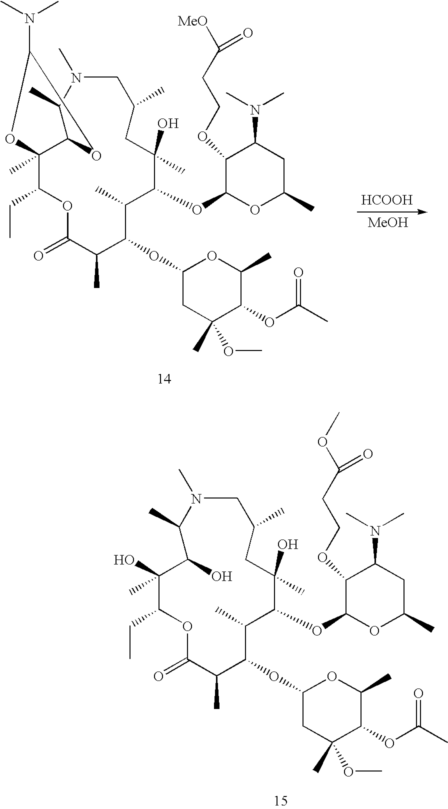

Water (3 mL) and 20 drops of HCOOH (conc.) were added to a solution of Compound 14 from step 1 (2.8 g) in MeOH (50 mL). The reaction mixture was stirred at 60° C. for 15 h when MeOH was evaporated yielding crude title product (3.2 g)

MS (ES+): 877.5 [MH]⁺

### Step 3: Preparation of Intermediate 4

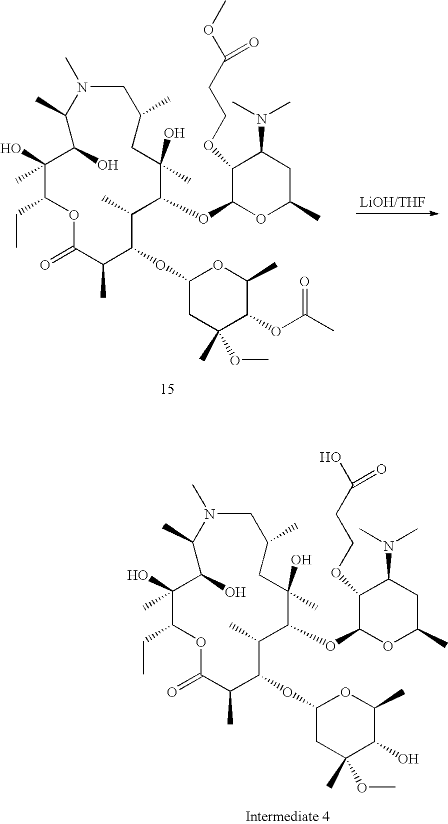

To the solution of Compound 15 (3.2 g) in THF (50 mL) 0.5M LiOH was added (25 mL). The reaction was stirred at r.t. for 48 hours, then the solvent was evaporated. This yielded crude product (2.78 g) which was purified at SPE column (50 g), yielding the title product (130.7 mg).

MS (ES+): 821.4 [MH]+

### Intermediate 5

### N-(7-chloro-4-quinolinyl)glycine

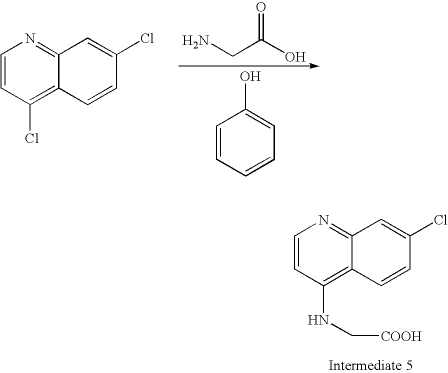

4,7-Dichloroquinoline (4.4 g, 22.22 mmol), glycine (3.34 g, 44.4 mmol) and phenol (12.02 g, 128 mmol) were placed into reaction flask and reaction mixture was stirred at 120° C. for 18 hours, then additional 1 eq. of glycine (1.668 g, 22.22 mmol) was added. Reaction mixture was stirred for next 3 hours and then cooled at r.t. and diluted with EtOAc. Precipitate was filtered off, washed with EtOAc and then dissolved in 10% Na₂CO₃ (supported by heating). Into this solution toluene (5 mL) was added, solution cooled at 5° C. and pH adjusted to 5.7 (by 6M HCl). Precipitate was filtered off, washed with water and toulene, dried under reduced pressure at 60° C. over night to yield the titled product (3.75 g).

MS (ES+): 237 [MH]⁺

¹³C-NMR (DMSO) δ/ppm: 171.35, 151.98, 150.72, 149.29, 134.12, 127.84, 124.86, 124.15, 117.98, 99.80, 45.27.

### EXAMPLES

### Example 1

### 2′-O-[3-({4-[(7-chloro-4-quinolinyl)amino]butanoyl}amino)propyl]-9-deoxo-9a-methyl-9a-aza-9a-homoerythromycin A

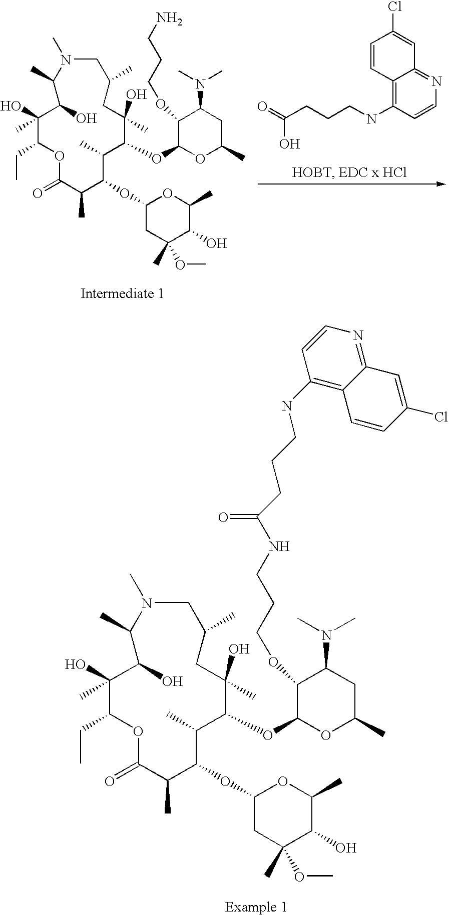

To a solution of Intermediate 1 (0.18 g, 0.22 mmol) in CH₂Cl₂ (10 ml), 4-[(7-chloro-4-quinolinyl)amino]butanoic acid (0.066 g, 0.25 mmol), HOBt (0.037 g, 0.286 mmol), 1-(3-dimethylaminopropyl)-3-ethylcarbodiimide (0.071 g, 0.37 mmol) and Et₃N (0.25 ml) were added and the reaction mixture was stirred overnight at r.t. Water was added, the organic layer separated, pH of water layer was adjusted to 6.5 and extracted with DCM. To the water layer DCM was added, pH adjusted to 9.0 and extracted with DCM. DCM layers at pH 6.5 and 9.0 were combined and dried over Na₂SO₄. DCM was evaporated affording 0.25 g of the title product as a light yellow powder.

MS (ES+): 1052.7 [MH]⁺;

¹³C-NMR (CDCl₃) δ/ppm: 179.0, 173.5, 151.7, 149.8, 147.2, 136.1, 126.8, 125.8, 123.2, 117.5, 103.1, 98.0, 94.5, 82.7, 79.8, 78.3, 77.6, 77.3, 74.6, 73.9, 73.4, 73.2, 72.2, 70.2, 68.3, 65.6, 65.1, 62.9, 49.4, 45.5, 44.5, 44.2, 42.5, 40.6, 38.4, 36.3, 34.7, 34.6, 29.0, 28.9, 27.6, 26.8, 22.3, 23.0, 21.8, 21.5, 18.3, 16.4, 14.8, 11.4, 8.5, 7.5.

### Example 2

### 2′-O-[3-({4-[(7-chloro-4-quinolinyl)amino]butanoyl}amino)propyl]-3-O-decladinosyl-9-deoxo-9a-methyl-9a-aza-9a-homoerythromycin A

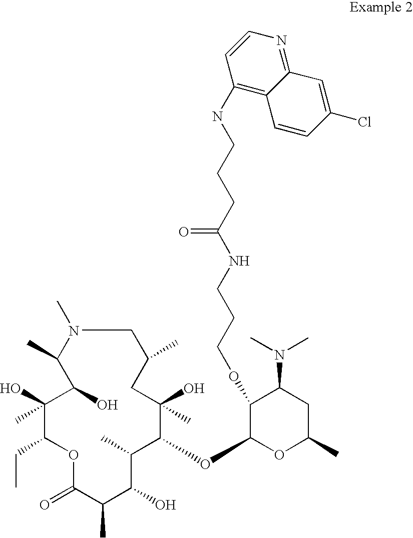

Example 1 (0.24 g, 0.2 mmol) was dissolved in 0.5 M HCl (30 mL) and stirred at r.t. for 17 hours. The reaction mixture was diluted with water and organic product extracted with DCM (5×25 mL), pH of the water layer was adjusted to pH 9.5 and extracted with DCM. Organic extracts (ph 9.5) were dried over Na₂SO₄ and solvent evaporated yielding 0.18 g of the title product as light yellow crystals.

MS (ES+): 894.5 [MH]⁺

### Example 3

### 2′-O-{3-[(7-Chloro-4-quinolinyl)amino]propyl}-9-deoxo-9a-methyl-9a-aza-9a-homoerythromycin A

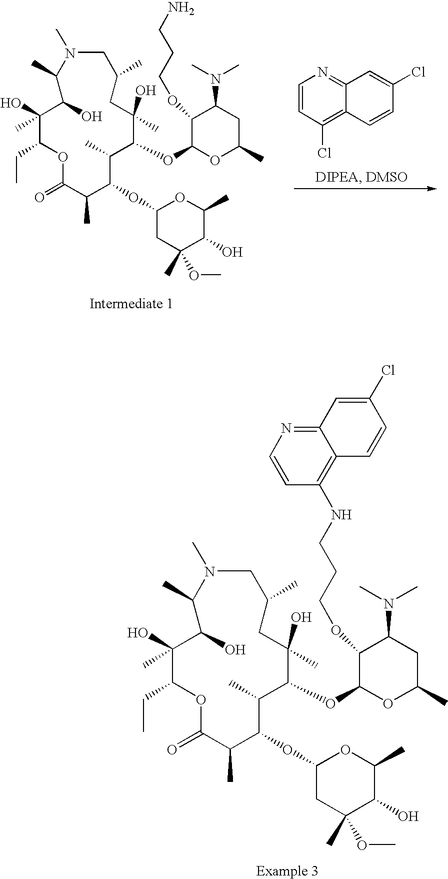

### Procedure A

To a solution of Intermediate 1 (4.28 g, 5.3 mmol) in DMSO (35 mL), 4,7-dichloroquinoline (3.3 g, 16.7 mmol) and diisopropylethyl amine (2.5 mL) were added. The reaction mixture was heated at 110° C. for 20 hours. Reaction mixture was cooled to r.t., diluted with EtOAc and washed with water. Combined organic layers were dried over Na₂SO₄ and organic solvent evaporated. Crude product was purified by column chromatography (DCM:MeOH:NH₄OH 90:9:1.5) yielding 1.2 g of the title product as a light yellow crystals.

MS (ES+): 967.6 [MH]⁺.

¹³C-NMR (CDCl₃) δ/ppm: 178.69, 151.22, 150.54, 147.72, 135.38, 127.32, 125.07, 122.51, 117.44, 102.79, 98.49, 94.36, 82.47, 80.21, 77.89, 77.16, 77.03, 74.29, 73.67, 73.11, 72.39, 70.01, 68.07, 65.67, 64.68, 62.59, 49.41, 45.41, 42.61, 42.36, 42.23, 40.88, 36.31, 34.73, 30.28, 28.44, 27.58, 26.68, 22.14, 21.61, 21.33, 21.29, 18.34, 16.25, 14.59, 11.24, 8.60, 7.52.

### Procedure B

### 2′-O-{3-[(7-Chloro-4-quinolinyl)amino]propyl}-9-deoxo-9a-methyl-9a-aza-9a-homoerythromycin A

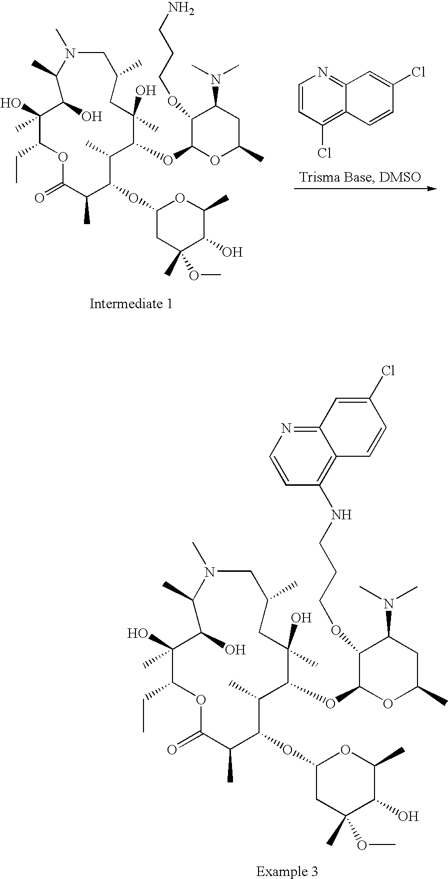

To a solution of Intermediate 1 (25.0 g, 31 mmol) in DMSO (250 mL), 4,7-dichloroquinoline (30.7 g, 155 mmol) and Trisma Base (18.77 g, 155 mmol) were added. The reaction mixture was heated at 105° C. for 18 hours. Reaction mixture was cooled to r.t. and evaporated yielding slurry product. The slurry product was resolved in dichloromethane (500 ml) and water (1500 ml). The pH of the mixture was adjusted to 5.0 by addition of 1 M HCl and layers were separated. The water layer was extracted at pH 5 with DCM (5×500 ml). The layers were separated. A further amount of DCM (500 ml) was added and pH adjusted to 6.0 by addition of 1M NaOH; layers were separated. The water layer was extracted at pH 6.0-6.5 with DCM (22×500 ml). The organic layers were dried over Na₂SO₄ and the solvent evaporated yielding crude product (17.913 g) as light yellow crystals. The crude product (17.91 g) was re-crystallized from acetonitrile to afford the title product (12.89 g).

MS (ES+): 967.6 [MH]⁺.

¹³C-NMR (DMSO) δ/ppm: 177.39, 152.09, 150.45, 149.42, 133.60, 127.87, 124.37, 124.29, 117.86, 102.34, 98.98, 94.67, 82.65, 80.10, 77.73, 77.40, 76.69, 75.23, 73.95, 73.19, 72.84, 69.952, 68.959, 67.12, 65.07, 64.31, 61.85, 49.17, 45.14, 42.01, 41.85, 41.31, 40.38, 36.04, 35.00, 32.43, 28.87, 27.77, 26.20, 22.38, 21.77, 21.38, 21.31, 18.89, 18.04, 15.10, 11.32, 8.70, 7.07.

### Example 4

### 11-O-Acetyl-2′-O-{3-[(7-Chloro-4-quinolinyl)amino]propyl}-3-O-decladinosyl-9-deoxo-9a-methyl-9a-aza-9a-homoerythromycin A

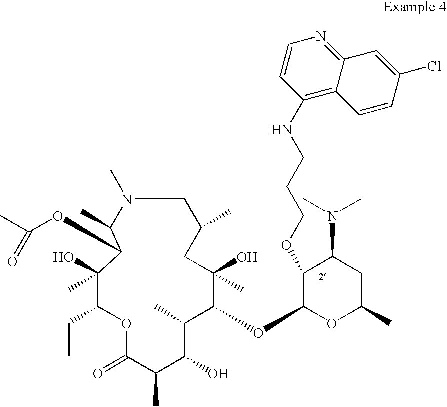

To a solution of Intermediate 2 (0.5 g, 0.5 mmol) in DMSO (5 mL), 4,7-dichloroquinoline (0.55 g, 2.76 mmol) and diisopropylethyl amine (0.3 mL) were added. The reaction mixture was heated at 100° C. for 4 hours and then for an additional 17 hours at r.t. The reaction mixture was diluted with water and extracted with EtOAc. Combined organic layers were dried over anhydrous Na₂SO₄, and organic solvent evaporated yielding 0.83 g of yellow oil residue which was dissolved in 0.25 M HCl and stirred at r.t. After 17 h the precipitate was filtered off and the mother liquor was extracted with DCM (5×25 mL) and pH of water layer was adjusted to pH 9.5. Water layer (pH 9.5) was extracted with DCM, organic extracts at pH 9.5 dried over Na₂SO₄ and solvent evaporated yielding 0.115 g of title product as light yellow powder.

MS (ES+): 852 [MH]⁺

### Example 5

### 2′-O-{3-[(7-Chloro-4-quinolinyl)amino]propyl}-3-O-decladinosyl-9-deoxo-9a-methyl-9a-aza-9a-homoerythromycin A

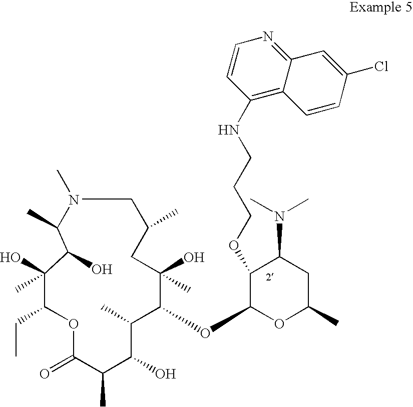

### Procedure A

To the solution of Example 4 (0.235 g, 0.28 mmol) in THF (10 mL), LiOH (1 mL, 0.5 M in water) was added. Reaction mixture was stirred at 65° C. for 5 hours and then 7 days at r.t. The reaction mixture was then diluted with water and product was extracted with EtOAc.

Combined EtOAc layers were dried over Na₂SO₄ and solvent evaporated yielding title product (0.222 g) as a yellow oil product.

MS (ES⁺): 809.03 [MH]⁺;

### Procedure B

Example 3 (0.6 g, 0.6 mmol) in HCl (40 mL, 3M) was stirred at r.t. for 1 hour. The reaction mixture was diluted with water, pH adjusted to pH 9.24 and extracted with DCM. Combined organic layers were dried over Na₂SO₄. After evaporating of solvent and purification by column chromatography (DCM:MeOH:NH₄OH=90:9:1.5) title product (0.54 g) was obtained as a white powder.

MS (ES⁺): 809.03 [MH]⁺.

¹³C-NMR (CDCl₃) δ/ppm: 177.44, 150.94, 150.79, 147.84, 135.37, 127.51, 125.29, 122.30, 117.33, 102.95, 98.51, 89.37, 79.05, 78.93, 77.64, 75.61, 74.23, 73.09, 71.59, 70.96, 68.83, 65.48, 62.65, 44.35, 42.27, 41.84, 41.84, 41.58, 40.83, 37.03, 35.77, 29.86, 28.44, 26.59, 26.34, 21.33, 21.06, 20.91, 16.14, 16.08, 10.91, 7.79, 7.61

### Procedure C

A solution of Example 3 (0.53 g, 0.5 mmol) in HCl (20 mL, 3M) was stirred at r.t. for 1.5 hour. The reaction mixture was diluted with water, pH adjusted to pH 9.0 and extracted with DCM. Combined organic layers were washed with water (five times) and dried over Na₂SO₄. The title product (0.301 g) as a white powder was obtained after evaporating the solvent.

MS (ES⁺): 809.03 [MH]⁺.

¹³C-NMR (DMSO) δ/ppm: 175.86, 152.17, 150.47, 149.42, 133.59, 127.91, 124.37, 124.33, 117.81, 100.68, 98.84, 84.34, 79.73, 76.73, 76.55, 76.40, 73.87, 72.71, 69.76, 69.20, 67.88, 64.23, 61.84, 43.59, 41.48, 41.00, 36.37, 35.72, 35.51, 31.32, 29.09, 26.66, 25.94, 22.43, 21.61, 21.54, 20.98, 18.14, 16.87, 14.33, 10.86, 8.37, 6.57.

### Example 6

### 2′-O-{3-[(4-quinolinyl)amino]propyl}-9-deoxo-9a-methyl-9a-aza-9a-homoerythromycin A

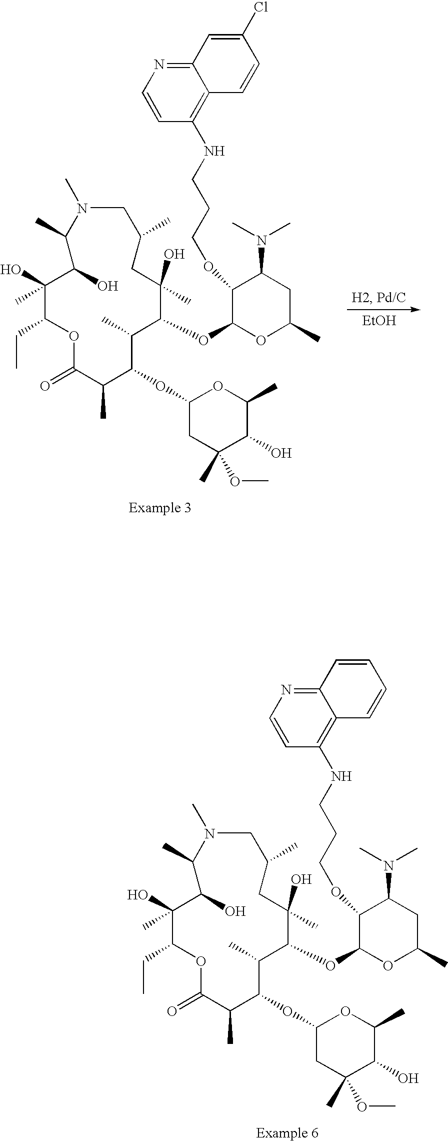

10% Pd/C catalyst (75 mg, 0.07 mmol) was added to an ethanol solution (25 mL) of Example 3 (150 mg, 0.15 mmol) and the reaction mixture was stirred under a hydrogen atmosphere (5 barr). After 4 hours the catalyst was removed by filteration and the solvent was evaporated yielding crude product (130 mg) which was further purified by column chromatography (eluent DCM:MeOH:NH₄OH=90:9:1.5) yielding the title product (90 mg) as a white powder.

MS (ES⁺): 933.6 [MH]⁺.

¹³C-NMR (DMSO-d6) δ/ppm: 177.42, 150.83, 150.33, 148.59, 129.33, 128.95, 124.01, 121.94, 119.25, 102.36, 98.45, 94.69, 82.65, 80.13, 77.74, 77.4, 76.71, 75.23, 73.97, 73.21, 72.89, 70.09, 68.93, 67.16, 65.06, 64.38, 61.79, 49.18, 45.14, 42.09, 41.90, 41.29, 40.38, 36.07, 35.01, 32.23, 29.04, 27.81, 26.28, 22.40, 21.79, 21.39, 21.33, 18.90, 18.09, 15.11, 11.33, 8.78, 7.11.

### Example 7

### 2′-O-[3-({2-[(7-chloro-4-quinolinyl)amino]ethanoyl}amino)propyl]-9-deoxo-9a-methyl-9a-aza-9a-homoerythromycin A

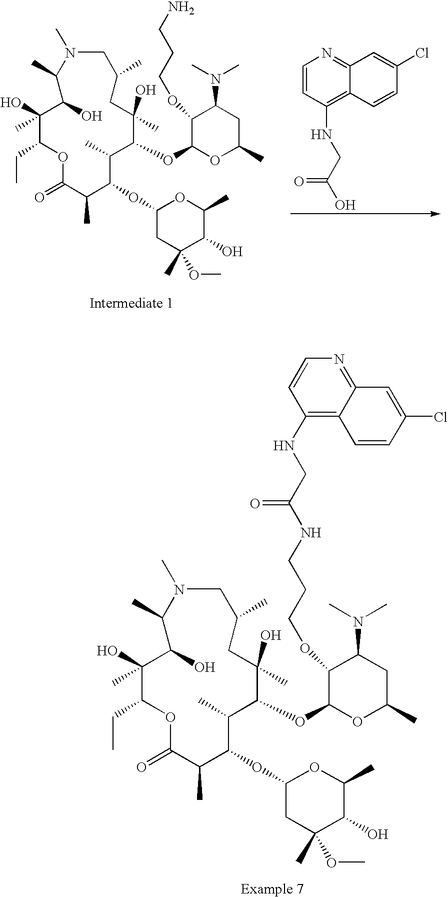

PS-Carbodiimide resin (PS-CDI, loading: 1.2 mmol/g) (325 mg, 0.403 mmol) was added to a dry reaction vessel. Intermediate 5 (77 mg, 0.326 mmol) and 1-hydroxybenzotriazole hydrate (29.3 mg, 0.217 mmol), dissolved in a mixture of DCM (5 mL) and DMF (2.5 mL), were added to the dry resin. The mixture was stirred at r.t. for 5 minutes then Intermediate 1 (250 mg, 0.310 mmol), dissolved in DCM (5 mL) was added. The reaction mixture was heated by microwave irradiation at 70° C. for 6 minutes.

HOBt was scavenged using PS-trisamine (loading: 4.11 mmol/g) (420 mg, 1.73 mmol) for 3 hours at room temperature. Product was filtered off and the resin washed with DCM (2×10 mL). After evaporating of filtrate white foam (278 mg) was obtained. Crude material was dissolved in EtOAc (3 mL) and precipitated with addition of n-hexane. Isolated precipitate was further recrystallised from acetone/petroleter and the title product (68 mg) was isolated. Further crystalisation from filtrate resulted in isolation of additional amount of the title product (85 mg).

MS (ES⁺): 1024 [MH]⁺.

¹³C-NMR (DMSO-d6) δ/ppm: 177.45, 168.63, 152.13, 150.56, 149.31, 133.84, 127.86, 124.67, 124.51, 117.89, 102.41, 99.43, 94.78, 82.66, 80.22, 77.75, 77.52, 76.76, 75.23, 74.02, 73.2, 72.95, 70.36, 68.96, 67.18, 65.06, 64.24, 61.77, 49.21, 46.21, 45.18, 42.21, 41.90, 41.16, 37.00, 36.09, 35.03, 31.81, 30.23, 27.85, 26.39, 22.47, 21.77, 21.38, 18.90, 18.06, 15.17, 11.34, 8.80, 7.20.

### Example 8

### 2′-O-[3-({2-[(7-chloro-4-quinolinyl)amino]ethanoyl}amino)propyl]-3-O-decladinosyl-9-deoxo-9a-methyl-9a-aza-9a-homoerythromycin A

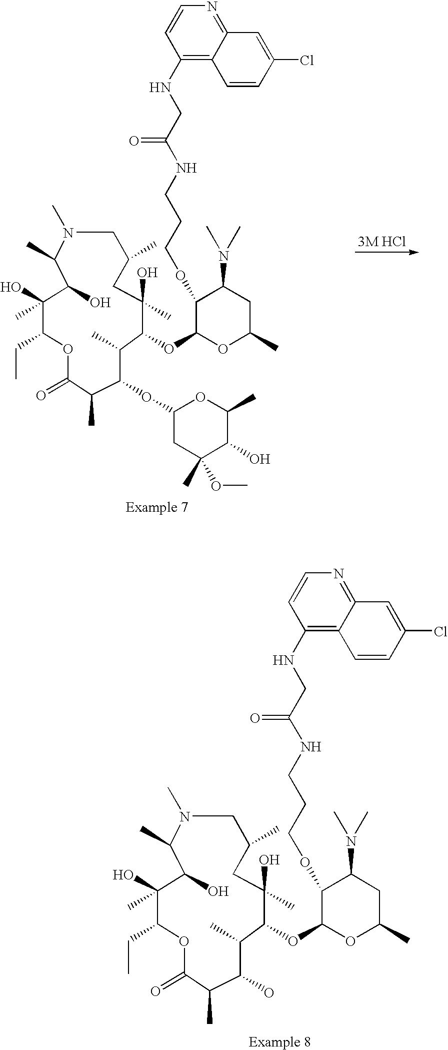

Example 7 (0.08 g, 0.081 mmol) in HCl (10 mL, 3M) was stirred at r.t. for 30 minutes. The reaction mixture was diluted with EtOAc (20 mL), pH adjusted to pH 9.5 (by addition of 6M NaOH) and layers were separated. Organic extracts were washed with water (2×20 mL). Combined organic layers were evaporated yielding the title product (50 mg).

MS (ES⁺): 866.59 [MH]⁺.

¹³C-NMR (DMSO-d6) δ/ppm: 175.97, 168.63, 152.2, 150.52, 149.32, 133.82, 127.87, 124.68, 124.50, 117.89, 100.71, 99.47, 84.29, 79.83, 76.84, 76.55, 76.53, 74.01, 72.82, 70.12, 69.18, 67.89, 64.13, 61.81, 46.19, 43.67, 41.25, 41.02, 37.18, 36.49, 35.87, 32.66, 30.27, 26.72, 26.17, 21.81, 21.56, 21.01, 18.21, 16.89, 10.91, 8.58, 6.69.

### Example 9

### 2′-O-[3-({2-[(4-quinolinyl)amino]ethanoyl}amino)propyl]-9-deoxo-9a-methyl-9a-aza-9a-homoerythromycin A

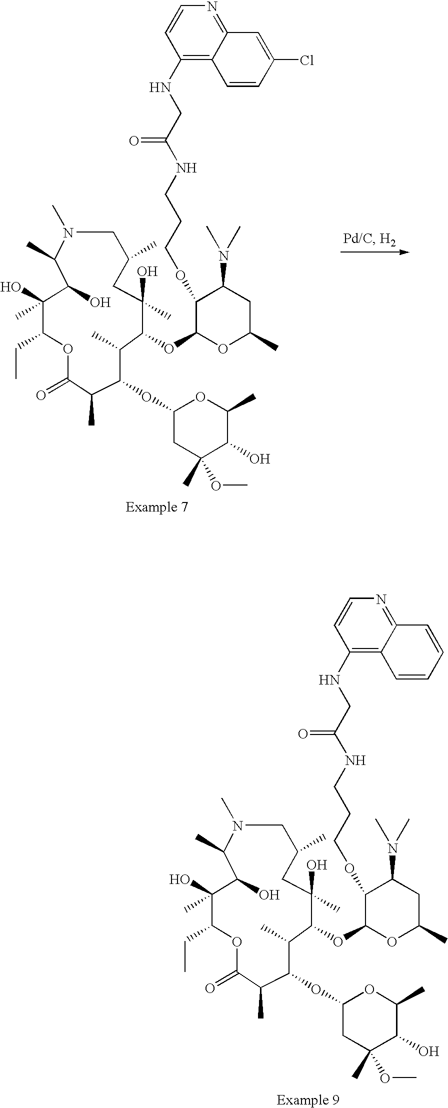

10% Pd/C catalyst (60 mg, 0.056 mmol) was added to an ethanol solution of Example 7 (200 mg, 0.195 mmol in 30 mL) and reaction mixture was stirred under hydrogen atmosphere (4 barr). After 4 hours the catalyst was filtered off, EtOAc (20 mL) and water (20 mL) were added to the filtrate and pH was adjusted to 4 (1M HCl). Water layer was then extracted with DCM (2×30 mL) and layers separated. By addition of 1M NaOH pH of water layer was adjusted to 6.5, extracted with DCM (2×30 mL) and layers separated. Organic layers at pH 6.5 were combined and water was added. By addition of NH₄OH pH was adjusted at pH 9.5, layers separated. Crude product obtained after evaporation of the solvent was recrystallised from diisopropyl-ether yielding the title product (120 mg).

MS (ES⁺): 990.6 [MH]⁺.

¹³C-NMR (DMSO-d6) δ/ppm: 176.86, 168.26, 150.31, 149.73, 147.98, 128.80, 128.58, 123.81, 121.47, 118.68, 101.83, 98.39, 94.24, 82.10, 79.66, 77.21, 76.98, 76.21, 74.66, 73.45, 72.62, 72.41, 69.82, 68.37, 66.61, 64.50, 63.66, 61.20, 48.63, 45.74, 44.63, 41.65, 41.32, 40.57, 36.43, 35.52, 34.47, 31.23, 29.68, 27.27, 25.83, 21.89, 21.19, 20.82, 20.78, 18.33, 17.49, 14.59, 10.76, 8.25, 6.64.

### Example 10

### 2′-O-[3-({2-[(4-quinolinyl)amino]ethanoyl}amino)propyl]-3-O-decladinosyl-9-deoxo-9a-methyl-9a-aza-9a-homoerythromycin A

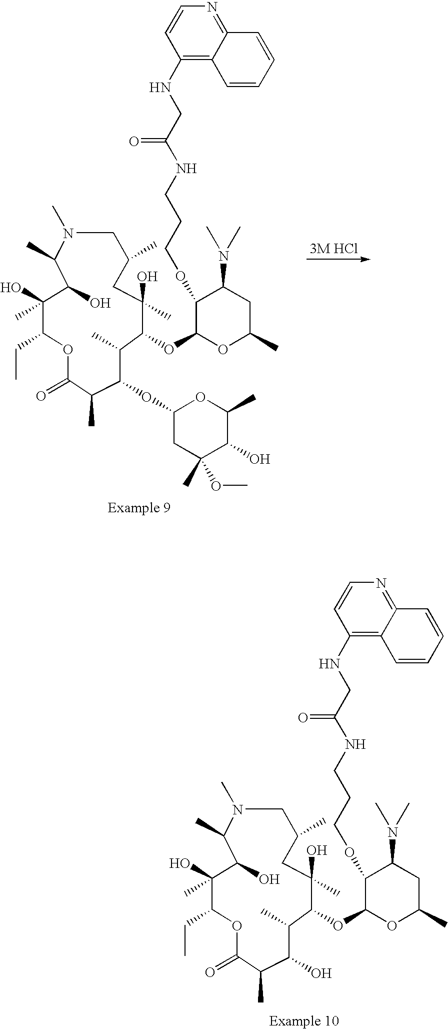

Example 9 (0.08 g, 0.081 mmol) in HCl (10 mL, 3M) was stirred at r.t. for 30 minutes. The reaction mixture was diluted with EtOAc (20 mL), pH adjusted to pH 9.5 by addition of 6M NaOH and layers were separated. Organic extracts were washed with water (2×20 mL). Combined organic layers were evaporated yielding the crude title product (58 mg).

MS (ES⁺): 832.5 [MH]⁺; LC-MS (Area %): 85.9.

10% Pd/C catalyst (5 mg, 0.0047 mmol) was added to an ethanol solution of crude product (58 mg in 20 mL) and the reaction mixture was stirred under hydrogen atmosphere (4 barr). After 1.5 hours the catalyst was filtered off and the solvent evaporated. Recrystalisation of the crude product (eluent ethyl-acetate/n-hexane followed by aceton/petroleum-ether) yielded the title product (37 mg).

MS (ES⁺): 832.57 [MH]⁺; LC-MS (Area %): 94.1.

¹³C-NMR (DMSO-d6) δ/ppm: 175.98, 168.89, 150.79, 150.41, 148.34, 129.26, 129.22, 124.46, 122.07, 119.19, 100.67, 99.00, 84.29, 79.65, 76.74, 76.54, 74.00, 72.86, 70.06, 67.86, 64.17, 61.93, 46.29, 43.68, 41.19, 37.08, 36.54, 35.88, 32.55, 30.28, 26.66, 26.13, 23.18, 21.79, 21.53, 21.00, 18.21, 16.89, 10.91, 8.59, 6.71.

### Example 11

### 2′-O-[3-({2-[(7-chloro-4-quinolinyl)amino]ethyl}amino)-3-oxopropyl]9-deoxo-9a-methyl—9a-aza-9a-homoerythromycin A

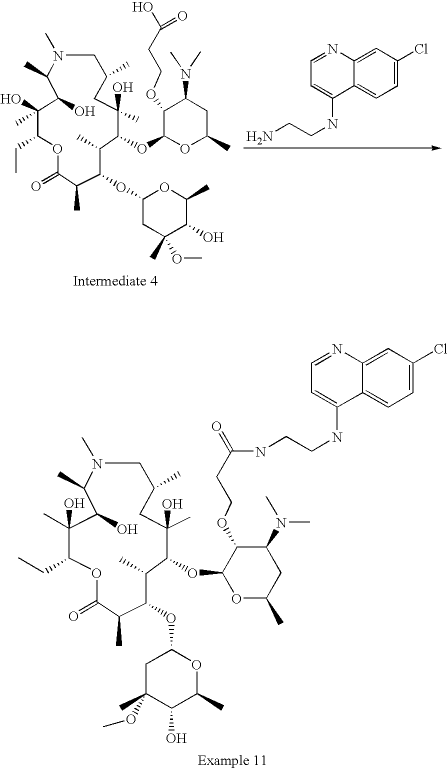

To a solution of Intermediate 4 (95 mg, 0.1157 mmol) in DCM (10 ml) triethylamine (0.113 ml, 0.81192 mmol), HOBt (18 mg, 0.1319 mmol), Intermediate 3 (1 eq., 0.10149 mmol) and EDC×HCl (33 mg, 0.1725 mmol) were added. Reaction mixture was stirred at r.t. for 42 hours. To the reaction mixture water (30 mL) was added (pH 8.0) and extracted with DCM (3×30 ml). Organic layers were collected and dried on Na₂SO₄. Solvent was evaporated to afford 130.7 mg of yellowish solid which was purified on Isolute SPE 10 g column eluting with CH₂Cl₂/[MeOH/NH₄OH] 90/[9:1.5] (70/0→70/1.36→70/2.72→70/4.0→70/5.4→70/6.8→70/8.1 resulting in the title product (62 mg).

MS (ES+) m/z: 1024.69 [MH]+

¹³C-NMR (DMSO-d6) δ/ppm: 176.8, 171.5, 151.6, 149.8, 148.8, 133.1, 127.3, 123.9, 123.4, 117.1, 101.7, 98.3, 94.1, 82.3, 78.9, 77.1, 76.9, 76.1, 74.7, 73.4, 72.6, 72.3, 68.4, 67.4, 66.6, 64.5, 63.5, 61.2, 48.5, 44.5, 42.3, 41.6, 41.3, 40.3, 40.3, 37.7, 36.4, 35.5, 34.4, 30.0, 27.2, 28.8, 21.8, 21.1, 20.8, 19.9, 18.3, 17.4, 14.5, 10.7, 8.1, 6.6.

### Example 12

### 2′-O-[3-({4-[(4-quinolinyl)amino]butanoyl}amino)propyl]-9-deoxo-9a-methyl-9a-aza-9a-homoerythromycin A

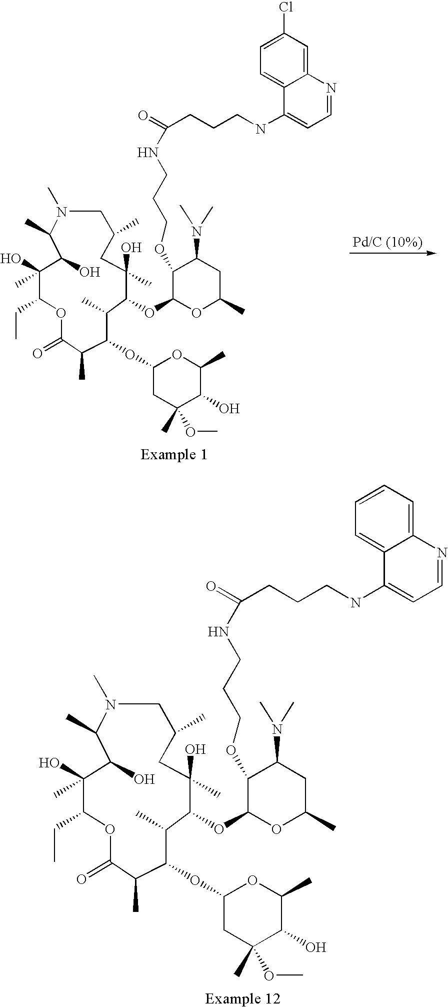

To a solution of Example 1 (288 mg, 0.27 mmol) in ethanol (30 ml), 10% Pd/C catalyst (60 mg) was added and the reaction mixture was stirred at r.t. under 3.2 barr of H₂-pressure for 5.5 hours. The catalyst was filtered off, solvent was evaporated and residue dissolved in water (80 ml) and DCM. By addition of 10% NaOH pH value was adjusted to 9.6 and product was extracted with DCM (2×100 ml). Collected organic layers were dried over Na₂SO₄ and solvent was evaporated to yield the title product (186 mg) as white powder.

MS (ES+): 1018.78 [MH]⁺

¹³C-NMR (DMSO-d6) δ/ppm: 176.9, 171.5, 150.5, 149.8, 148.2, 128.9, 128.5, 123.6, 121.5, 118.8, 101.9, 97.9, 94.3, 82.2, 79.6, 77.3, 77.1, 76.3, 74.8, 73.5, 72.7, 72.5, 69.9, 66.7, 64.6, 63.8, 61.3, 48.7, 44.7, 42.1, 41.4, 40.6, 36.4, 35.6, 34.6, 32.9, 29.6, 27.3, 25.9, 23.8, 21.9, 21.3, 20.9, 18.4, 17.6, 14.7, 10.8, 8.3.

### Example 13

### 2′-O-[3-[(3-quinolinylcarbonyl)amino]propyl]-9-deoxo-9a-methyl-9a-aza-9a-homoerythromycin A

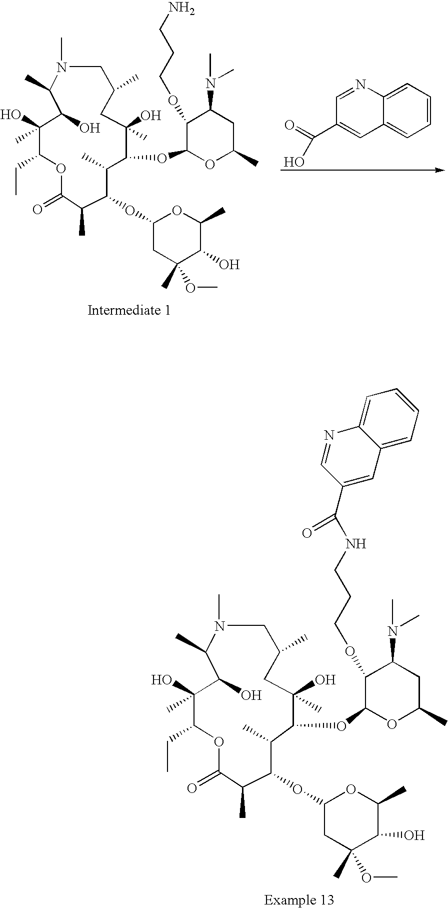

PS-Carbodiimide resin (PS-CDI, loading: 1.2 mmol/g) (38.8 mg, 0.048 mmol) was added to a dry reaction vessel. 3-Quinolinecarboxylic acid (6.75 mg, 0.039 mmol) and HOBt (3.5 mg, 0.026 mmol), dissolved in a mixture of DCM (1.2 mL) and DMF (0.2 mL), was added to the dry resin. The mixture was stirred at r.t. for 5 minutes, then Intermediate 1 (30 mg, 0.037 mmol), dissolved in DCM (1.2 mL), was added. The reaction mixture was heated by microwave irradiation at 70° C. for 6 minutes.

HOBt was scavenged using PS-trisamine (loading: 4.11 mmol/g) (31.63 mg, 0.13 mmol) for 3 hours at r.t. Product was filtered off and the resin washed with DCM (4×0.5 mL). The organic solvent was evaporated yielding the title product (26 mg).

MS (ES+): 961.4 [MH]⁺

¹³C-NMR (DMSO-d6) δ/ppm: 177.4, 165.0, 162.7, 149.1, 148.8, 135.6, 131.6, 129.4, 129.1, 127.9, 127.8, 127.0, 102.4, 94.7, 82.8, 80.3, 77.7, 77.5, 76.7, 75.2, 73.9, 73.2, 72.9, 70.7, 70.5, 68.8, 67.1, 65.1, 64.2, 61.7, 49.2, 45.2, 42.0, 41.9, 41.2, 37.6, 36.2, 36.0, 35.0, 32.4, 31.1, 30.2, 27.8, 26.2, 22.3, 21.8, 21.4, 21.3, 18.9, 18.0, 15.1, 11.3, 8.7, 7.0.

### Example 14

### 2′-O-{3-[(4-quinolinyl methyl)amino]propyl}-9-deoxo-9a-methyl-9a-aza-9a-homoerythromycin A

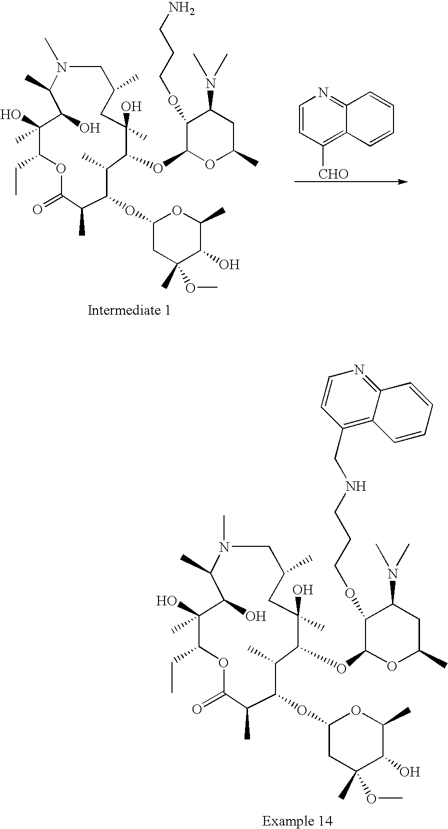

To a solution of Intermediate 1 (1 g, 1.24 mmol) in MeOH (35 mL) triethylamine (0.585 mL, 4.2 mmol) and 4-quinolinecarbaldehyde (164 mg, 1.04 mmol) were added, the reaction mixture was stirred at r.t. for 18 hours, then NaBH₄ (94 mg, 2.48 mmol) was added. The reaction mixture was stirred for a further 3 hours then the solvent was evaporated. The residue was dissolved in water, pH adjusted to 9.5, and extracted with DCM. Combined organic layers were dried over anhydrous Na₂SO₄. Evaporation of the solvent yielded 1.3 g of yellow powder product which was further purified by column chromatography (eluent DCM:MeOH:NH₄OH=90:9:1.5) yielded the title product (0.35 g).

MS (ES+): 947.66 [MH]⁺

¹³C-NMR (DMSO-d6) δ/ppm: 176.91, 149.97, 147.46, 146.26, 129.33, 128.76, 126.52, 126.01, 123.69, 119.46, 101.79, 94.18, 82.13, 79.49, 77.20, 76.95, 76.20, 74.70, 73.46, 72.63, 72.41, 70.26, 68.40, 66.6, 64.52, 63.79, 61.26, 48.98, 48.53, 46.76, 44.61, 41.63, 41.36, 40.59, 35.57, 34.48, 31.31, 29.55, 27.25, 25.81, 21.88, 21.23, 20.83, 20.76, 18.34, 17.53, 14.61, 10.80, 8.23, 6.67.

### Example 15

### 2′-O-{3-[(4-quinolinylmethyl)amino]propyl}-3-O-decladinosyl-9-deoxo-9a-methyl-9a-aza-9a-homoerythromycin A

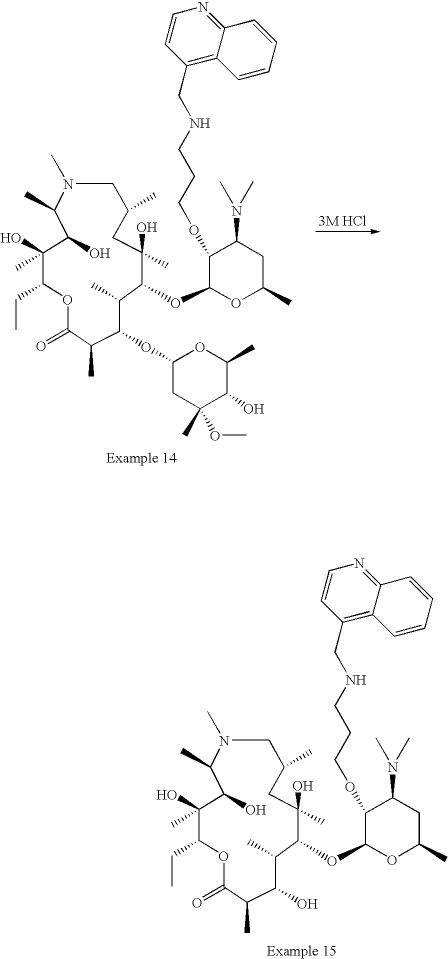

Example 14 (0.1 g, 0.1 mmol) in HCl (2.5 mL, 3M) was stirred at r.t. for 2 hours. The reaction mixture was diluted with water, pH adjusted to pH 9.5 and extracted with CH₂Cl₂. Organic extracts were washed with water (7×15 mL), dried over anhydrous Na₂SO₄. Evaporating of the solvent yielded the title product (93 mg) as a white powder.

MS (ES⁺): 789.5 [MH]⁺.

¹³C-NMR (DMSO-d6) δ/ppm: 175.31, 149.89, 147.36, 146.08, 129.23, 128.65, 126.42, 125.93, 123.60, 119.49, 100.07, 83.66, 79.03, 76.15, 75.93, 75.85, 73.33, 72.17, 69.77, 69.03, 67.24, 63.55, 61.17, 49.02, 46.69, 43.03, 40.60, 35.90, 35.22, 32.19, 29.78, 26.05, 25.46, 21.09, 20.90, 20.36, 17.46, 16.20, 10.23, 7.89, 6.01.

### Example 16

### 2′-O-{3-[methyl(4-quinolinyl methyl)amino]propyl}-9-deoxo-9a-methyl-9a-aza-9a-homoerythromycin A

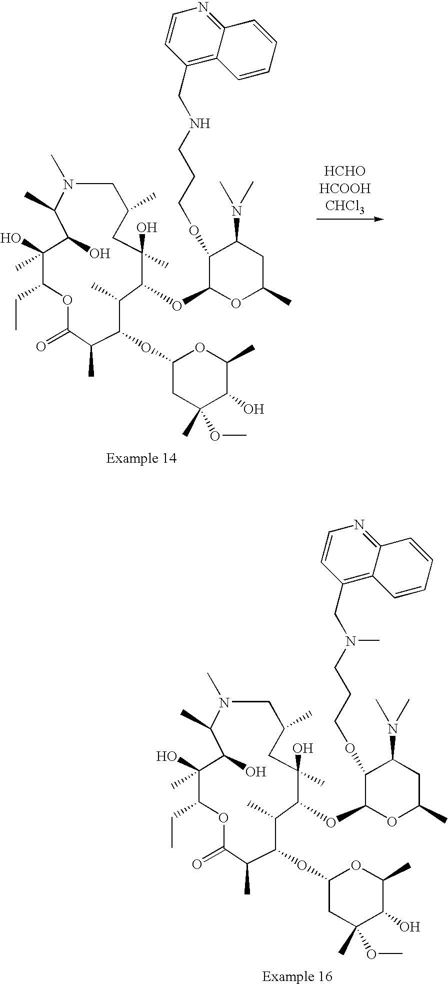

To a solution of Example 14 (0.15 g, 0.158 mmol) in chloroform (5 mL) formaldehyde (0.028 mL) and formic acid (0.149 mL, 4.05 mmol) were added. The reaction mixture was stirred at 60° C. for 18 hours, then diluted with DCM and water. Layers were separated, organic layer washed with brine and dried over Na₂SO₄. Evaporation of the solvent yielded crude product (0.16 g) which was further purified by column chromatography (eluent DCM:MeOH:NH₄OH=90:9:1.5) yielding the title product (0.1 g).

MS (ES+): 961.67 [MH]⁺

¹³C-NMR (DMSO-d6) δ/ppm: 177.06, 150.04, 147.92, 144.71, 129.44, 128.97, 127.14, 126.04, 124.51, 121.24, 101.96, 94.36, 82.17, 79.85, 77.44, 77.11, 76.45, 74.89, 73.68, 72.81, 72.62, 69.92, 68.55, 66.80, 64.71, 63.92, 61.45, 58.60, 54.39, 48.78, 44.81, 42.14, 41.89, 41.54, 40.96, 35.75, 34.71, 32.60, 27.80, 27.43, 25.99, 22.00, 21.81, 21.40, 21.04, 18.52, 17.75, 14.78, 10.98, 8.49, 6.90.

### Example 17

### 2′-O-{3-[(3-quinolinyl methyl)amino]propyl}-9-deoxo-9a-methyl-9a-aza-9a-homoerythromycin A

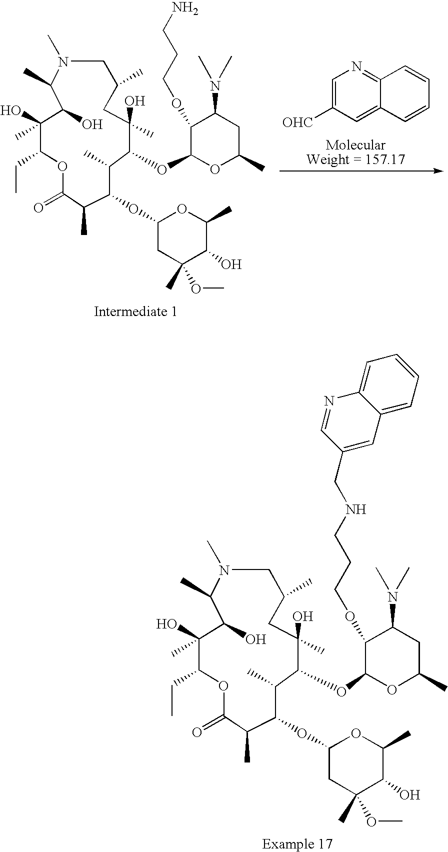

To a solution of Intermediate 1 (1 g, 1.24 mmol) in MeOH (35 mL) triethylamine (0.585 mL, 4.2 mmol) and 3-quinolinecarbaldehyde (164 mg, 1.04 mmol) were added, the reaction mixture was stirred at r.t. for 18 hours, then NaBH₄ (94 mg, 2.48 mmol) was added. The reaction mixture was stirred for a further 3 hours, then the solvent was evaporated. The residue obtained after evaporation of the solvent was dissolved in water, pH adjusted to 9.5, and extracted with DCM. Combined organic layers were dried over anhydrous Na₂SO₄. Evaporation of the solvent yielded a yellow powder (1.2 g) which was further purified by column chromatography (eluent DCM:MeOH:NH₄OH=90:9:1.5) yielding the title product (0.14 g).

MS (ES+): 947.5 [MH]⁺

¹³C-NMR (DMSO-d6) δ/ppm: 176.88, 151.36, 146.59, 133.62, 128.57, 128.47, 127.52, 127.37, 126.33, 101.76, 94.18, 82.14, 79.50, 77.18, 76.95, 76.16, 74.69, 73.43, 72.59, 72.38, 70.18, 68.34, 66.56, 64.49, 63.72, 61.20, 50.35, 48.51, 46.24, 44.59, 41.61, 41.31, 40.56, 35.53, 34.45, 31.47, 29.44, 27.22, 25.79, 21.86, 21.20, 20.94, 20.79, 18.31, 17.49, 14.62, 10.76, 8.18, 6.62.

### Example 18

### 2′-O-{3-[methyl(3-quinolinyl methyl)amino]propyl}-9-deoxo-9a-methyl-9a-aza-9a-homoerythromycin A

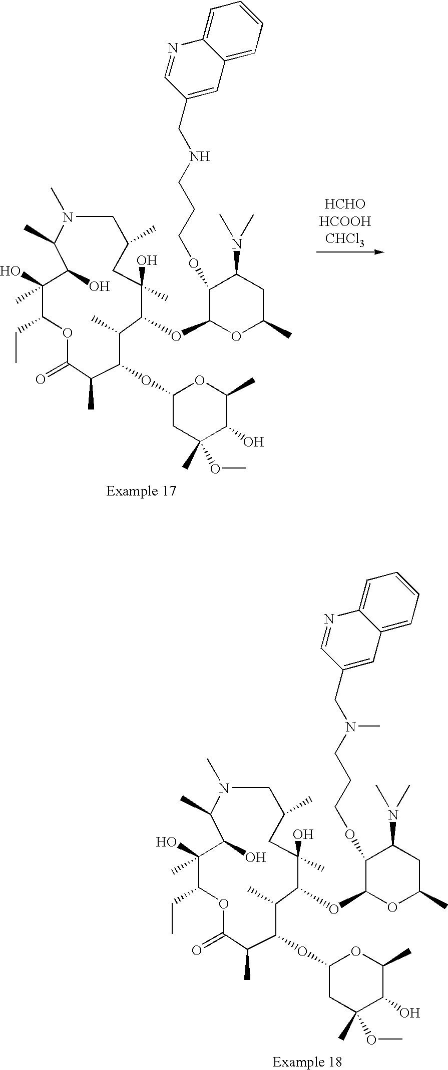

To a solution of Example 17 (340 mg, 0.359 mmol) in chloroform (12 mL) formaldehyde (0.053 ml, 1.920 mmol) and formic acid (0.281 ml, 7.34 mmol) were added. The reaction mixture was stirred at 60° C. for 18 hours, then diluted with DCM and water. By addition of 1M NaOH pH was adjusted to 6.5 and layers were separated. To the organic layer water was added and by addition of NH₄OH pH was adjusted to 9.5. Layers were separated, solvent evaporated yielding crude product (0.23 g) which was recrystallised from ether/n-hexane. Product was filtered off yielding the title product (0.115 g).

MS (ES+): 961.87 [MH]⁺

¹³C-NMR (DMSO-d6) δ/ppm: 177.40, 152.11, 147.27, 135.12, 132.48, 129.27, 129.03, 128.12, 127.85, 126.94, 102.30, 94.73, 82.60, 80.26, 77.75, 77.46, 76.72, 75.22, 73.97, 73.15, 72.95, 70.24, 68.90, 67.09, 65.06, 64.22, 61.77, 59.37, 54.35, 49.07, 45.14, 42.26, 42.16, 41.35, 41.35, 36.10, 36.10, 35.00, 33.11, 28.22, 27.81, 26.34, 22.43, 21.76, 21.35, 21.35, 18.90, 18.00, 15.19, 11.32, 8.85, 7.18.

### Example 19

### 2′-O-{3-[(3-quinolinylmethyl)amino]propyl}-3-O-decladinosyl-9-deoxo-9a-methyl-9a-aza-9a-homoerythromycin A

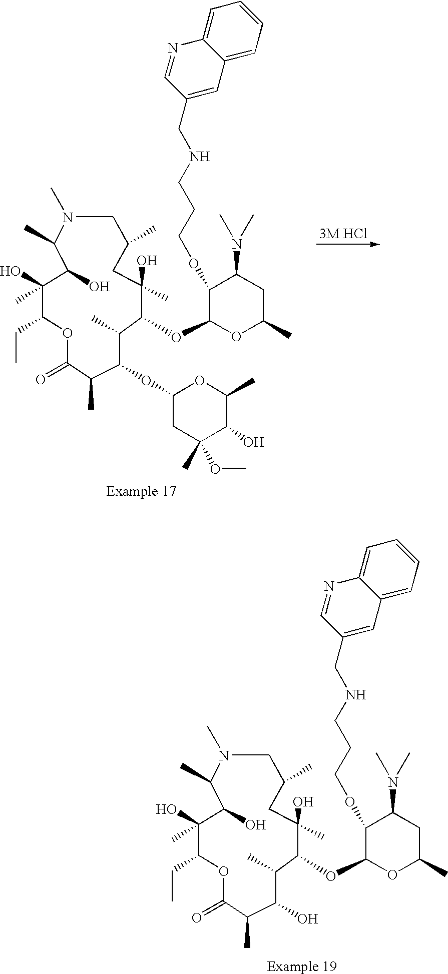

Example 17 (0.14 g, 0.148 mmol) in HCl (10 mL, 3M) was stirred at r.t. for 1 hour. The reaction mixture was diluted with EtOAc, pH adjusted to pH 9.5 (addition of 6M NaOH) and layers separated. Organic extracts were washed with water (7×20 mL), solvent evaporated. Crude product (91 mg) was recrystallised from ether/n-hexane yielding the title product (74 mg).

MS (ES⁺): 789.63 [MH]⁺.

¹³C-NMR (DMSO-d6) δ/ppm: 175.54, 151.46, 146.67, 133.84, 133.67, 128.66, 128.60, 127.62, 127.49, 126.42, 100.27, 83.80, 79.26, 76.35, 76.12, 76.04, 73.53, 72.36, 69.89, 68.72, 67.43, 63.68, 61.34, 50.63, 46.30, 43.21, 40.86, 40.86, 40.86, 36.03, 35.41, 32.74, 29.97, 26.27, 25.63, 21.27, 21.11, 20.56, 17.61, 16.43, 10.45, 8.07, 6.29

### Example 20

### 2′-O-{3-[(2-quinolinyl methyl)amino]propyl}-9-deoxo-9a-methyl-9a-aza-9a-homoerythromycin A

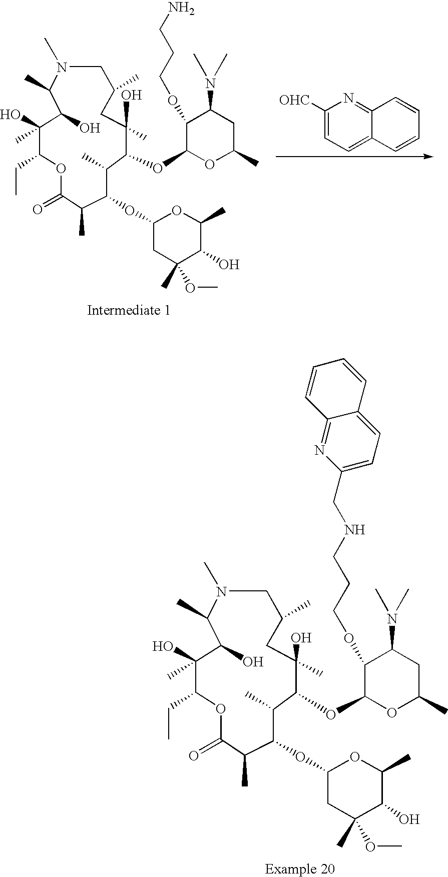

To the solution of Intermediate 1 (1 g, 1.24 mmol) in MeOH (35 mL) triethylamine (0.585 mL, 4.2 mmol) and 2-quinolinecarbaldehyde (164 mg, 1.04 mmol) were added, the reaction mixture was stirred at r.t. for 18 hours, then NaBH₄ (94 mg, 2.48 mmol) was added. Reaction mixture was stirred for a further 2 hours, then the solvent was evaporated. The residue obtained was dissolved in water, pH adjusted to 9.5, and extracted with DCM. Combined organic layers were dried over anhydrous Na₂SO₄. Evaporation of the solvent yielded a yellow powder (1.3 g) which was further purified by column chromatography (eluent DCM:MeOH:NH₄OH=90:9:1.5) yielding the title product (0.16 g).

MS (ES+): 947.78 [MH]⁺

¹³C-NMR (DMSO-d6) δ/ppm: 176.85, 160.40, 146.76, 135.96, 129.19, 128.18, 127.58, 126.71, 125.75, 120.18, 101.72, 94.19, 82.14, 79.54, 77.20, 76.97, 76.20, 74.70, 73.46, 72.59, 72.41, 70.23, 68.39, 66.60, 64.51, 63.73, 61.22, 54.75, 48.57, 46.54, 44.58, 41.64, 41.28, 40.45, 35.53, 34.49, 31.22, 29.29, 27.19, 25.82, 21.84, 21.18, 20.79, 18.29, 17.49, 14.57, 10.74, 8.20, 6.64.

### Example 21

### 2′-O-{3-[(3-chloro-1-isoquinolinyl)amino]propyl}-9-deoxo-9a-methyl-9a-aza-9a-homoerythromycin A

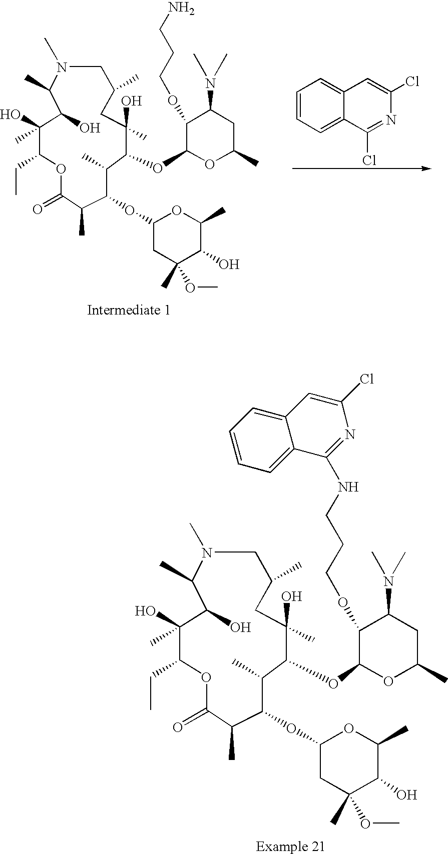

To a solution of 1,3-dichloroisoquinoline (100 mg, 0.505 mmol) in DMSO (10 ml) tris(hydroxymethyl)aminoethane (306 mg, 2.52 mmol) and Intermediate 1 were added. The reaction mixture was stirred at 100° C. for 7 hours followed by stirring at r.t. over night. The reaction mixture was then diluted with EtOAc (20 mL) and washed with water (40 mL). Water was added (20 mL) to the organic layer and pH adjusted to 5 by addition of 1MHCl. The water layer was extracted with DCM (2×30 mL). To organic extracts at pH 5 water was added and pH adjusted to 9.5 by addition of NH₄OH. Organic extracts at pH 9.5 were evaporated yielding crude product (0.3 g) which was recrystallised from acetone/petrol ether yielding the title product (0.273 g).

MS (ES+): 967.78 [MH]⁺

¹³C-NMR (DMSO-d6) δ/ppm: 177.49, 156.09, 144.29, 138.70, 130.82, 126.25, 125.87, 123.55, 116.85, 106.57, 102.4, 94.63, 82.60, 80.22, 77.75, 77.38, 76.69, 75.21, 73.95, 73.18, 72.86, 70.34, 68.99, 67.12, 65.03, 64.16, 61.80, 49.22, 45.18, 42.05, 41.95, 41.35, 39.12, 36.04, 35.01, 32.75, 29.50, 27.83, 26.23, 22.34, 21.78, 21.39, 21.34, 18.90, 18.07, 15.10, 11.34, 8.67, 7.08.

### Example 22

### 2′-O-{3-[methyl(3-quinolinylmethyl)amino]propyl}-3-O-decladinosyl-9-deoxo-9a-methyl-9a-aza-9a-homoerythromycin A

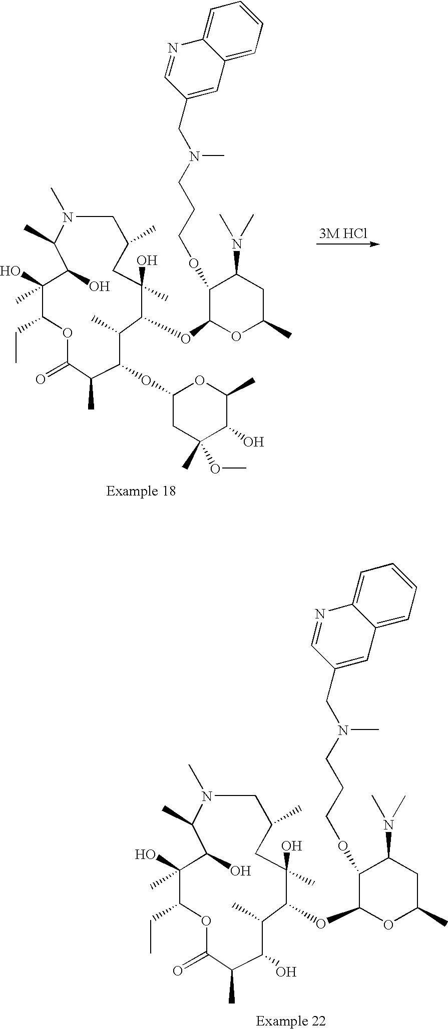

Example 18 (70 mg, 0.073 mmol) in HCl (10 mL, 3M) was stirred at r.t. for 30 minutes. The reaction mixture was diluted with EtOAc, pH adjusted to pH 9.5 (addition of 6M NaOH) and layers separated. Organic extracts were washed with water (2×20 mL), and the solvent evaporated. Crude product (37 mg) was recrystallised from petrol ether yielding the title product (25 mg).

MS (ES⁺): 803.69 [MH]⁺.

¹³C-NMR (DMSO-d6) δ/ppm: 175.97, 152.08, 147.24, 135.17, 132.42, 129.32, 129.034, 128.14, 127.84, 126.98, 100.73, 84.21, 79.83, 76.79, 76.57, 76.48, 73.96, 72.82, 70.00, 67.89, 64.17, 61.74, 59.31, 54.47, 43.65, 42.19, 41.52, 41.12, 36.49, 35.86, 34.06, 28.30, 26.73, 26.09, 21.71, 21.55, 20.96, 17.88, 16.86, 10.89, 8.64, 6.75.

### In Vitro Assay

The in vitro potency of the compounds has been compared with that of azithromycin. Using the methodology described in the In vitro screening protocol the compounds listed in the Table 1 were profiled for their antimalarial activity against two different P. falciparum parasites (W2 and 3D7A) with different susceptibilities. The IC₅₀ values of the tested compounds are provided as a range:

Key to Table

| | |
|---|---|
| A | X ≦ 100 |
| B | 100 < X ≦ 200 |
| C | 200 < X ≦ 1000 |
| D | 1000 < X ≦ 2500 |
| E | 2500 < X ≦ 3000 |
| F | 3000 < X ≦ 3500 |
| G | 3500 < X ≦ 5000 |
| H | 5000 < X ≦ 10000 |

X = IC₅₀ in ng/mL

TABLE 1

In vitro screening protocol II

| Compound | W2 | 3D7A |
|---|---|---|
| azithromycin | E | H, >H |
| Example 1 | A | A |
| Example 2 | B | B |
| Example 3 | A | A |
| Example 4 | A | A |
| Example 5 | A | A |
| Example 6 | A | A |
| Example 7 | A | A |
| Example 8 | B | A |
| Example 9 | A | B |
| Example 10 | C | D |
| Example 11 | A | A |
| Example 12 | B | C |

1. A compound of Formula (I): 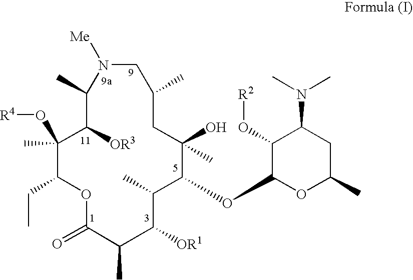
wherein
R¹ represents H or a α-L-cladinosyl group of Formula (a) 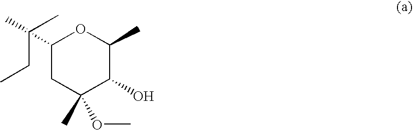
R² represents the formula —(CH₂)ₐ—X—(CH₂)_{b}—(NH)_{c}-A;
R³ represents H or —C(O)C₁₋₃alkyl or R³ and R⁴ taken together with the intervening atoms form a cyclic carbonate group of Formula (b): 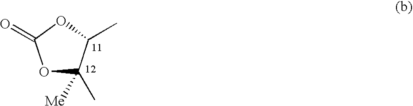
R⁴ represents H or R³ and R⁴ taken together with the intervening atoms form a cyclic carbonate group of Formula (b);
X represents —N(R⁵)—, —NHC(O)— or —C(O)NH—;
R⁵ represents H or C₁₋₃alkyl;
A represents a moiety of Formula (c) or (d): 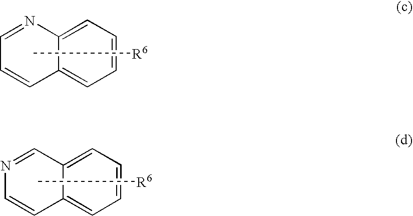
attached to the rest of the molecule through any available carbon atom;
R⁶ represents H or halogen and is attached to Formula (c) or (d) at any available carbon atom;
a is an integer from 2 to 6;
b is an integer from 0 to 6;
c is 0 or 1;
provided that when c is 1 then b is an integer from 1 to 6;
or salts thereof.

2. A compound or a salt thereof as claimed in claim 1, wherein R¹ represents H.

3. A compound or a salt thereof as claimed in claim 1, wherein R¹ represents a α-L-cladinosyl group of Formula (a).

4. A compound or a salt thereof as claimed in claim 1, wherein X is NHC(O) and c is 1.

5. A compound or a salt thereof as claimed in claim 4, wherein a is 3 and b is 3 and A is a compound of Formula (c)

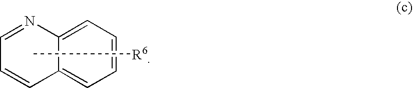

6. A compound or a salt thereof as claimed in claim 1, wherein X is NH, b is 0, c is 0, and A is a compound of Formula (c)

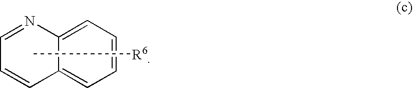

7. A compound of Formula (I) selected from the group consisting of:
2′-O-[3-({4-[(7-chloro-4-quinolinyl)amino]butanoyl}amino)propyl]-9-deoxo-9a-methyl-9a-aza-9a-homoerythromycin A;
2′-O-[3-({4-[(7-chloro-4-quinolinyl)amino]butanoyl}amino)propyl]-3-O-decladinosyl-9-deoxo-9a-methyl-9a-aza-9a-homoerythromycin A;
2′-O-{3-[(7-Chloro-4-quinolinyl)amino]propyl}-9-deoxo-9a-methyl-9a-aza-9a-homoerythromycin A;
11-O-Acetyl-2′-O-{3-[(7-Chloro-4-quinolinyl)amino]propyl}-3-O-decladinosyl-9-deoxo-9a-methyl-9a-aza-9a-homoerythromycin A;
2′-O-{3-[(7-Chloro-4-quinolinyl)amino]propyl}-3-O-decladinosyl-9-deoxo-9a-methyl-9a-aza-9a-homoerythromycin A;
2′-O-{3-[(4-quinolinyl)amino]propyl}-9-deoxo-9a-methyl-9a-aza-9a-homoerythromycin A;
2′-O-[3-({2-[(7-chloro-4-quinolinyl)amino]ethanoyl}amino)propyl]-9-deoxo-9a-methyl-9a-aza-9a-homoerythromycin A;
2′-O-[3-({2-[(7-chloro-4-quinolinyl)amino]ethanoyl}amino)propyl]-3-O-decladinosyl-9-deoxo-9a-methyl-9a-aza-9a-homoerythromycin A;
2′-O-[3-({2-[(4-quinolinyl)amino]ethanoyl}amino)propyl]-9-deoxo-9a-methyl-9a-aza-9a-homoerythromycin A;
2′-O-[3-({2-[(4-quinolinyl)amino]ethanoyl}amino)propyl]-3-O-decladinosyl-9-deoxo-9a-methyl-9a-aza-9a-homoerythromycin A;
2′-O-[3-({2-[(7-chloro-4-quinolinyl)amino]ethyl}amino)-3-oxopropyl]9-deoxo-9a-methyl-9a-aza-9a-homoerythromycin A;
2′-O-[3-({4-[(4-quinolinyl)amino]butanoyl}amino)propyl]-9-deoxo-9a-methyl-9a-aza-9a-homoerythromycin A;
2′-O-[3-[(3-quinolinylcarbonyl)amino]propyl]-9-deoxo-9a-methyl-9a-aza-9a-homoerythromycin A;
2′-O-{3-[(4-quinolinylmethyl)amino]propyl}-9-deoxo-9a-methyl-9a-aza-9a-homoerythromycin A;
2′-O-{3-[(4-quinolinylmethyl)amino]propyl}-3-O-decladinosyl-9-deoxo-9a-methyl-9a-aza-9a-homoerythromycin A;
2′-O-{3-[methyl(4-quinolinylmethyl)amino]propyl}-9-deoxo-9a-methyl-9a-aza-9a-homoerythromycin A;
2′-O-{3-[(3-quinolinylmethyl)amino]propyl}-9-deoxo-9a-methyl-9a-aza-9a-homoerythromycin A;
2′-O-{3-[methyl(3-quinolinylmethyl)amino]propyl}-9-deoxo-9a-methyl-9a-aza-9a-homoerythromycin A;
2′-O-{3-[(3-quinolinylmethyl)amino]propyl}-3-O-decladinosyl-9-deoxo-9a-methyl-9a-aza-9a-homoerythromycin A;
2′-O-{3-[(2-quinolinylmethyl)amino]propyl}-9-deoxo-9a-methyl-9a-aza-9a-homoerythromycin A;
2′-O-{3-[(3-chloro-1-isoquinolinyl)amino]propyl}-9-deoxo-9a-methyl-9a-aza-9a-homoerythromycin A; and
2′-O-{3-[methyl(3-quinolinylmethyl)amino]propyl}-3-O-decladinosyl-9-deoxo-9a-methyl-9a-aza-9a-homoerythromycin A; and
salts thereof.

8. 2′-O-{3-[(7-Chloro-4-quinolinyl)amino]propyl}-9-deoxo-9a-methyl-9a-aza-9a-homoerythromycin A, or a salt thereof.

9. 2′-O-{3-[(7-Chloro-4-quinolinyl)amino]propyl}-3-O-decladinosyl-9-deoxo-9a-methyl-9a-aza-9a-homoerythromycin A, or a salt thereof.

10. A compound of Formula (II) 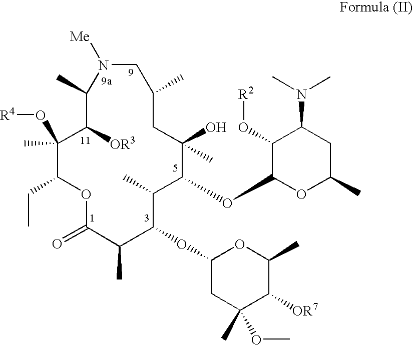
wherein
R² is aminopropyl
R³ is H or C(O)CH₃;
R⁴ represents H;
R⁷ is H or 3-aminopropyl;
or a salt thereof.

11. A compound of Formula (II) as claimed in claim 10, selected from:
2′-O-(3-Aminopropyl)-9-deoxo-9a-methyl-9a-aza-9a-homoerythromycin A; or
11-O-Acetyl-2′-O, 4″-O-di-(3-aminopropyl)-9-deoxo-9a-methyl-9a-aza-9a-homo-erythromycin A, or a salt thereof.

12. A compound or a salt thereof as claimed in claim 1, wherein the salt is a pharmaceutically acceptable salt.

13. Process for the preparation of compounds of Formula (I) according to claim 1 wherein R¹ is a group of Formula a), X is divalent radical —N(R⁵)—, a is 2-6, b is 1-6 and c is 0 comprising reductive amination of a compound of Formula (III) 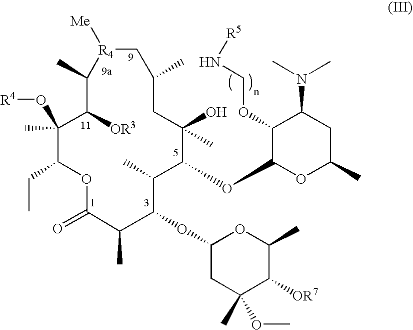
with a suitable aldehyde of formula (IV)

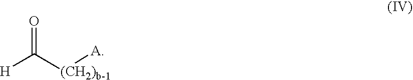

14. Use of a compound of Formula (II) according to claim 10 as an intermediate for the preparation of compounds of Formula (I).

15. A method for the therapeutic and/or prophylactic treatment of malaria in a subject in need of such treatment comprising administering to the subject a therapeutically effective amount of compound of Formula (I) according to claim 1 or a pharmaceutically acceptable salt thereof.

16. The method of claim 15, wherein the subject has been infected with Plasmodium falciparum, Plasmodium vivax, Plasmodium ovale or Plasmodium malariae.

17. A pharmaceutical composition comprising a compound according to claim 1, or a pharmaceutically acceptable salt thereof, in association with at least one pharmaceutically acceptable carrier.
